(19) United States
(12) Patent Application Publication
(10) Pub. No.: US 2022/0054864 A1
(43) Pub. Date: Feb. 24, 2022

(54) NONINVASIVE ELECTRICAL TREATMENT DEVICES

(71) Applicant: Baldoni Neuromodulation LLC, Austin, TX (US)

(72) Inventor: Daniel Baldoni, Austin, TX (US)

(21) Appl. No.: 17/309,676

(22) PCT Filed: Dec. 17, 2019
(86) PCT No.: PCT/US19/66819

§ 371 Date: Jun. 14, 2021

Related U.S. Application Data
(60) Provisional application No. 62/782,679, filed on Dec. 20, 2018.

(51) Int. Cl. A61N 7/02

(52) U.S. Cl. CPC A61N 7/02; A61N 2007/0021

(57) ABSTRACT

Noninvasive neuromodulation combines transcutaneous electrical modulation with heat and/or focused ultrasonic energy. A noninvasive neuromodulation device includes a first bipole electrode pair aligned along a first axis and a second bipole electrode pair aligned along a second axis, the first axis and the second axis defining a plane. A focused ultrasound (FUS) transducer can direct a focused ultrasound beam along a third axis that intersects the plane. A controller is electrically coupled to the first and second bipole electrode pairs and to the focused ultrasound transducer. The controller is configured to apply electrical energy having a frequency of between about 1 Hz to about 100 MHz to the first and second bipole electrode pairs, and to cause the FUS transducer to emit a focused ultrasound beam having a frequency of between about 20 kHz to about 10 MHz.

### CROSS-REFERENCE TO RELATED APPLICATION(S)

The present application is a 371 U.S. national phase application of International Patent Application No. PCT/US19/66819, filed Dec. 17, 2019, which claims the benefit of priority to U.S. Provisional Application No. 62/782,679, filed Dec. 20, 2018, each of which are hereby incorporated by reference in their entirety.

### TECHNICAL FIELD

The present technology relates to noninvasive neuromodulation and associated systems and methods of use. In particular, the present technology is directed to devices that apply electrical current to noninvasively modulate nerve activity.

### BACKGROUND

Implantable neuromodulation devices have been developed to treat pain, movement disorders, functional disorders, spasticity, cancer, cardiac disorders, and various other medical conditions. Implantable neuromodulation systems generally have an implantable signal generator and one or more leads that deliver electrical pulses to neurological tissue or muscle tissue. Use of these devices requires invasive surgical procedures that risk damage to surrounding tissue and involve extended recovery periods. Noninvasive neuromodulation systems avoid many of the problems associated with surgical procedures for implantable devices, but tend to be less effective in treating pain or other conditions. Accordingly, there is a need for improved systems and methods for noninvasive neuromodulation.

### SUMMARY

The present technology is directed to noninvasive neuromodulation systems and methods. The subject technology is illustrated, for example, according to various aspects described below, including with reference to FIGS. 1-13. Various examples of aspects of the subject technology are described as numbered clauses (1, 2, 3, etc.) for convenience. These are provided as examples and do not limit the subject technology. These clauses can be combined in any order and in any combination.

Clause 1. A noninvasive neuromodulation device comprising: a first bipole electrode pair aligned along a first axis; a second bipole electrode pair aligned along a second axis, the first axis and the second axis defining a plane; a focused ultrasound (FUS) transducer configured to direct a focused ultrasound beam along a third axis that intersects the plane; a controller electrically coupled to the first and second bipole electrode pairs and to the focused ultrasound transducer, the controller configured to: apply electrical energy having a frequency of between about 1 Hz to about 100 MHz to the first and second bipole electrode pairs; and cause the FUS transducer to emit a focused ultrasound beam having a frequency of between about 20 kHz to about 10 MHz.

Clause 2. The device of any one of the preceding Clauses, wherein the third axis is substantially orthogonal to the plane.

Clause 3. The device of any one of the preceding Clauses, wherein the third axis intersects the plane at an angle greater than 45 degrees.

Clause 4. The device of any one of the preceding Clauses, further comprising a third bipole electrode pair aligned along a fourth axis, wherein the controller is configured to apply electrical energy having a frequency of between about 1 Hz to about 100 MHz to the third bipole electrode pair.

Clause 5. The device of any one of the preceding Clauses, wherein the controller is configured to cause the FUS transducer to direct a focused ultrasound beam having a pulse repetition frequency of between about 0-10 kHz.

Clause 6. The device of any one of the preceding Clauses, wherein the first bipole electrode pair comprises first and second electrodes separated by a distance of between about 3-35 cm.

Clause 7. The device of any one of the preceding Clauses, wherein the first bipole electrode pair comprises first and second electrodes separated by a distance of between about 5-15 cm.

Clause 8. The device of any one of the preceding Clauses, wherein the controller is configured to apply electrical energy having a frequency of between about 1-300 kHz to the first and second bipole electrode pairs.

Clause 9. The device of any one of the preceding Clauses, wherein the controller is configured to apply electrical energy having a frequency of between about 9-11 kHz to the first and second bipole electrode pairs.

Clause 10. The device of any one of the preceding Clauses, wherein the controller is configured to apply electrical energy having a frequency of between about 3-5 kHz to the first and second bipole electrode pairs.

Clause 11. The device of any one of the preceding Clauses, wherein the controller is configured to apply electrical energy having a frequency of between about 20-100 kHz to the first and second bipole electrode pairs.

Clause 12. The device of any one of the preceding Clauses, wherein the controller is configured to cause the FUS transducer to emit a focused ultrasound beam having a frequency of between about 100 kHz to about 10 MHz.

Clause 13. The device of any one of the preceding Clauses, wherein the controller is configured to cause the FUS transducer to emit a focused ultrasound beam having a frequency of between about 250-750 kHz.

Clause 14. The device of any one of the preceding Clauses, wherein the focused ultrasound beam has a focal zone defined by full width at half maximum acoustic pressure field that is between about 25-90 mm from the plane.

Clause 15. The device of any one of the preceding Clauses, wherein the focused ultrasound beam has a focal zone defined by full width at half maximum acoustic pressure field with a focal zone width of between about 5-15 mm.

Clause 16. The device of any one of the preceding Clauses, wherein the focused ultrasound beam has a focal zone defined by full width at half maximum acoustic pressure field with a focal zone depth of between about 2-10 mm.

Clause 17. The device of any one of the preceding Clauses, wherein the FUS transducer is a spherical FUS transducer.

Clause 18. The device of any one of the preceding Clauses, wherein the spherical FUS transducer has a radius of curvature of between about 10-150 mm.

Clause 19. The device of any one of the preceding Clauses, wherein the FUS transducer is a cylindrical FUS transducer.

Clause 20. The device of any one of the preceding Clauses, further comprising an electromagnetic coil configured to induce a magnetic field directed substantially along the third axis, wherein the controller is electrically coupled to the electromagnetic coil and configured to apply electrical energy to the electromagnetic coil having a frequency of between about 1 Hz to about 100 Hz.

Clause 21. The device of any one of the preceding Clauses, wherein the controller is configured to electrical energy to the first and second bipole electrode pairs having the same frequency as the electrical energy applied to the electromagnetic coil.

Clause 22. The device of any one of the preceding Clauses, further comprising a temperature sensor electrically coupled to the controller, and wherein the controller, in response to an indication that the temperature sensor has detected a temperature above a predetermined threshold, is configured to: cease applying electrical energy to the first and second bipole pairs; and/or cease causing the FUS transducer to direct the focused ultrasound beam.

Clause 23. A noninvasive neuromodulation device comprising: an array of electrodes disposed around a central region, the electrodes configured to apply electrical current transcutaneously to a treatment site; a focused ultrasound (FUS) transducer disposed in the central region, the FUS transducer configured to apply focused ultrasonic energy to the treatment site; and a controller electrically coupled to the array of electrodes and to the FUS transducer, the controller configured to apply electrical energy to the array of electrodes and to cause the FUS transducer to emit a focused ultrasound beam.

Clause 24. The device of any one of the preceding Clauses, wherein the array of electrodes comprises at least four electrodes arranged circumferentially around the central region.

Clause 25. The device of any one of the preceding Clauses, wherein the array of electrodes comprises a plurality of bipole electrode pairs, with individual electrodes of each bipole electrode pair separated from one another by the central region.

Clause 26. The device of any one of the preceding Clauses, wherein the individual electrodes of each bipole electrode pair are separated from one another by a distance of between about 3-35 cm.

Clause 27. The device of any one of the preceding Clauses, wherein the individual electrodes of each bipole electrode pair are separated from one another by a distance of between about 5-15 cm.

Clause 28. The device of any one of the preceding Clauses, wherein the array of electrodes is arranged in a plane and configured to be disposed over a patient's skin adjacent to the treatment site.

Clause 29. The device of any one of the preceding Clauses, wherein the FUS transducer is configured to emit the focused ultrasound beam along an axis that intersects the plane.

Clause 30. The device of any one of the preceding Clauses, wherein the controller is configured to apply electrical energy to the array of electrodes having a frequency of between about 1 Hz to about 100 MHz.

Clause 31. The device of any one of the preceding Clauses, wherein the controller is configured to apply electrical energy to the array of electrodes having a frequency of between about 1-300 kHz.

Clause 32. The device of any one of the preceding Clauses, wherein the controller is configured to apply electrical energy to the array of electrodes having a frequency of between about 20-100 kHz.

Clause 33. The device of any one of the preceding Clauses, wherein the controller is configured to cause the FUS transducer to emit a focused ultrasound beam having a frequency of between about 100 kHz to about 10 MHz.

Clause 34. The device of any one of the preceding Clauses, wherein the controller is configured to cause the FUS transducer to emit a focused ultrasound beam having a frequency of between about 250-750 kHz.

Clause 35. The device of any one of the preceding Clauses, wherein the controller is configured to cause the FUS transducer to emit a focused ultrasound beam having a pulse repetition frequency of between about 0-10 kHz.

Clause 36. The device of any one of the preceding Clauses, wherein the focused ultrasound beam has a focal zone defined by full width at half maximum acoustic pressure field that is between about 25-90 mm from the plane.

Clause 37. The device of any one of the preceding Clauses, wherein the focused ultrasound beam has a focal zone defined by full width at half maximum acoustic pressure field with a focal zone width of between about 5-15 mm.

Clause 38. The device of any one of the preceding Clauses, wherein the focused ultrasound beam has a focal zone defined by full width at half maximum acoustic pressure field with a focal zone depth of between about 2-10 mm.

Clause 39. The device of any one of the preceding Clauses, wherein the FUS transducer is a spherical FUS transducer.

Clause 40. The device of any one of the preceding Clauses, wherein the spherical FUS transducer has a radius of curvature of between about 10-150 mm.

Clause 41. The device of any one of the preceding Clauses, wherein the FUS transducer is a cylindrical FUS transducer.

Clause 42. The device of any one of the preceding Clauses, further comprising an electromagnetic coil disposed in or over the central region and configured to induce a magnetic field directed toward the treatment site, wherein the controller is electrically coupled to the electromagnetic coil and configured to apply electrical energy to the electromagnetic coil having a frequency of between about 1 Hz to about 100 Hz.

Clause 43. The device of any one of the preceding Clauses, wherein the controller is configured to electrical energy to the array of electrodes having the same frequency as the electrical energy applied to the electromagnetic coil.

Clause 44. The device of any one of the preceding Clauses, further comprising a temperature sensor electrically coupled to the controller, and wherein the controller, in response to an indication that the temperature sensor has detected a temperature above a predetermined threshold, is configured to: cease applying electrical energy to the array of electrodes; and/or cease causing the FUS transducer to emit a focused ultrasound beam.

Clause 45. A noninvasive neuromodulation device comprising: an array of electrodes configured to apply electrical current transcutaneously to a treatment site, the array comprising: a first electrode bipole pair arranged along a first axis; a second electrode bipole pair arranged along a second axis intersecting the first axis; a controller electrically coupled to the array of electrodes, the controller configured to apply electrical energy to the array of electrodes at a frequency of at least about 200 Hz.

Clause 46. The device of any one of the preceding Clauses, wherein the array of electrodes are configured to deliver superficial heating to or adjacent to the treatment site.

Clause 47. The device of any one of the preceding Clauses, wherein the array of electrodes comprises at least four electrodes arranged circumferentially around a central region.

Clause 48. The device of any one of the preceding Clauses, wherein the controller is configured to apply electrical energy to the array of electrodes having a frequency of between about 200 Hz to about 100 MHz.

Clause 49. The device of any one of the preceding Clauses, wherein the controller is configured to apply electrical energy to the array of electrodes having a frequency of between about 1-300 kHz.

Clause 50. The device of any one of the preceding Clauses, wherein the controller is configured to apply electrical energy to the array of electrodes having a frequency of between about 20-100 kHz.

Clause 51. The device of any one of the preceding Clauses, further comprising a temperature sensor electrically coupled to the controller, and wherein the controller, in response to an indication that the temperature sensor has detected a temperature above a predetermined threshold, is configured to cease applying electrical energy to the array of electrodes.

Clause 52. The device of any one of the preceding Clauses, wherein the array of electrodes are carried by a housing configured to be disposed around a user's neck such that the electrodes face a rear surface of the user's neck.

Clause 53. A method for noninvasively modulating one or more nerves at a treatment site of a patient, the method comprising: applying electrical energy to a plurality of bipole electrode pairs disposed over a patient's skin proximate to the treatment site such that current flowing between each of the bipole electrode pairs intersects at or adjacent to the treatment site; and applying a focused ultrasound beam through the patient's skin and towards the treatment site.

Clause 54. The method of any one of the preceding Clauses, wherein applying the focused ultrasound beam through the patient's skin and towards the treatment site comprises heating the treatment site.

Clause 55. The method of any one of the preceding Clauses, wherein heating the treatment site lowers electrical impedance at the treatment site, thereby increasing electrical current density at the treatment site.

Clause 56. The method of any one of the preceding Clauses, wherein the treatment site is between about 30-80 mm below the patient's skin.

Clause 57. The method of any one of the preceding Clauses, wherein the treatment site comprises a dorsal root ganglion of the patient.

Clause 58. The method of any one of the preceding Clauses, wherein modulating one or more nerves at the treatment site treats back pain of the patient.

Clause 59. The method of any one of the preceding Clauses, wherein the treatment site comprises one or more of the sacral foramen of the patient.

Clause 60. The method of any one of the preceding Clauses, wherein modulating one or more nerves at the treatment site treats urinary incontinence of the patient.

Clause 61. The method of any one of the preceding Clauses, wherein the treatment site comprises one or more of thoracic vertebrae T5-T10 of the patient.

Clause 62. The method of any one of the preceding Clauses, wherein modulating one or more nerves at the treatment site treats symptoms of type 2 diabetes of the patient.

Clause 63. The method of any one of the preceding Clauses, wherein modulating one or more nerves at the treatment site treats symptoms of the patient associated with one or more of: chronic pain, gastroparesis, migraines, inflammatory bowel disease, rheumatoid arthritis, spinal cord injury, Parkinson's disease, essential tremor, dystonia, dementia, autism, Crohn's disease, ulcerative colitis, lupus, or multiple sclerosis.

Clause 64. The method of any one of the preceding Clauses, wherein applying the electrical energy and the focused ultrasound beam comprises applying the electrical energy and the focused ultrasound beam at a first location on the patient, the method further comprising: applying electrical energy to a second plurality of bipole electrode pairs disposed over the patient's skin at a second location proximate to the treatment site such that current flowing between each of the second plurality of bipole electrode pairs intersects at or adjacent to the treatment site; and applying a second focused ultrasound beam through the patient's skin at the second location and towards the treatment site.

Clause 65. The method of any one of the preceding Clauses, wherein the first and second locations are disposed on opposite sides of the patient's spine.

Clause 66. The method of any one of the preceding Clauses, wherein the electrical energy is alternating current.

Clause 67. The method of any one of the preceding Clauses, wherein the alternating current has a frequency of between about 1 Hz to about 100 MHz.

Clause 68. The method of any one of the preceding Clauses, wherein the alternating current has a frequency of between about 1-300 kHz.

Clause 69. The method of any one of the preceding Clauses, wherein the alternating current has a frequency of between about 20-100 kHz.

Clause 70. The method of any one of the preceding Clauses, wherein the bipole electrode pairs are arranged in a plane, and wherein applying the focused ultrasound beam comprises applying the focused ultrasound beam along an axis that intersects the plane.

Clause 71. The method of any one of the preceding Clauses, wherein the axis is substantially orthogonal to the plane.

Clause 72. The method of any one of the preceding Clauses, wherein bipole electrode pairs are arranged circumferentially around a central region, and wherein applying the focused ultrasound beam comprises applying the focused ultrasound beam from the central region, through the patient's skin, and towards the treatment site.

Clause 73. The method of any one of the preceding Clauses, wherein the focused ultrasound beam has a frequency of between about 20 kHz to about 10 MHz.

Clause 74. The method of any one of the preceding Clauses, wherein the focused ultrasound beam has a frequency of between about 250-750 kHz.

Clause 75. The method of any one of the preceding Clauses, wherein the focused ultrasound beam has a focal zone defined by full width at half maximum acoustic pressure field that is between about 30-80 mm beneath the patient's skin.

Clause 76. The method of any one of the preceding Clauses, wherein the focused ultrasound beam has a focal zone defined by full width at half maximum acoustic pressure field with a focal zone width of between about 5-15 mm.

Clause 77. The method of any one of the preceding Clauses, wherein the focused ultrasound beam has a focal zone defined by full width at half maximum acoustic pressure field with a focal zone width of between about 2-10 mm.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic diagram of a neuromodulation device in communication with a mobile computing device in accordance with embodiments of the present technology.

FIGS. 2A-2C are front, rear, and rear perspective views, respectively, of a neuromodulation device in accordance with embodiments the present technology.

FIGS. 3A and 3B are front and rear perspective views, respectively, of another neuromodulation device in accordance with embodiments of the present technology.

FIGS. 4A and 4B are top and side perspective views, respectively, of another neuromodulation device in accordance with embodiments of the present technology.

FIGS. 5A and 5B are top and side perspective views, respectively, of another neuromodulation device in accordance with embodiments of the present technology.

FIGS. 6A and 6B are top and side perspective views, respectively, of another neuromodulation device in accordance with embodiments of the present technology.

FIG. 7 illustrates another neuromodulation device in position over a spine of a patient, in accordance with embodiments of the present technology.

FIG. 8 illustrates a neuromodulation device for treatment of migraines, in accordance with embodiments of the present technology.

FIG. 9 illustrates another neuromodulation device for treatment of migraines, in accordance with embodiments of the present technology.

FIG. 10A illustrates a schematic side view of a neuromodulation device delivering electrical and ultrasonic energy to a treatment side in accordance with embodiments of the present technology.

FIG. 10B illustrates a schematic side view of another neuromodulation device delivering electrical and ultrasonic energy to a treatment site in accordance with embodiments of the present technology.

FIG. 11 illustrates anatomical targets for noninvasive modulation of the parasympathetic and sympathetic nervous systems.

FIG. 12 is a flow diagram of a method for noninvasively modulating one or more nerves at a treatment site of a patient, in accordance with embodiments of the present technology.

FIG. 13 is a flow diagram of another method for noninvasively modulating one or more nerves at a treatment site of a patient, in accordance with embodiments of the present technology.

### DETAILED DESCRIPTION

### I. Overview

Electrical modulation (e.g., stimulation or inhibition) of nerves can be used to treat a variety of conditions, including chronic pain, movement disorders, functional disorders, spasticity, cancer, cardiac disorders, and various other medical conditions. Electrical modulation can include stimulation (e.g., increasing nerve activity by triggering action potentials) and inhibition (e.g., decreasing activity of hyperactive nerves), depending on the current, frequency, and other parameters of the electrical energy supplied to the treatment site. For example, in some instances, electrical frequencies of between about 1 Hz to about 100 Hz can be used for muscle stimulation, electrical frequencies of between about 50 to about 300 Hz can be used for nerve stimulation, and electrical frequencies of between about 300 Hz and about 500 kHz can be used for nerve inhibition. Transcutaneous electrical nerve stimulation (TENS) and electrical muscle stimulation (EMS) are commonly used treatment modalities for the acute treatment of pain and has been proposed to have potential to treat a variety of other clinical applications. Commercial TENS or EMS devices include two or more electrodes that are adhered to the skin via conductive pads. The electrodes can be arranged in bipole pairs such that current principally flows from one electrode of a bipole pair to the other electrode of the bipole pair. These devices typically deliver electrical pulses at 0-100 Hz, with stimulation amplitudes in the range of 5-100 mA. Such transcutaneous electrical stimulation results in relatively shallow current penetration, with most of the electrical current concentrated near the surface of the skin. To increase current in tissue at a desired depth, interferential current TENS therapy uses 4 TENS electrodes arranged in an “X” fashion, which is believed to penetrate deeper structures and utilizes kHz frequency stimulation. Interferential current TENS therapy has been using to treat constipation and other gastrointestinal disorders and has been explored for stimulation of deep brain structures. This approach is thought to increase the current applied to a biological tissue volume because when current is only applied along one line of the “X” using a particular voltage, there is little or no current flowing in the other line of the “X” from that voltage. Applying two voltages across each line of the “X” increases the total current reaching a particular depth since now both lines of the “X” have current flowing through them. Even with inferential current TENS, however, it may be difficult or impossible to achieve the desired current density at a target site positioned beneath a patient's skin, for example at a dorsal root ganglion located 40-50 mm below the patient's skin.

Ultrasound energy can significantly reduce the electrical impedance of skin, muscle, connective tissue, and bone depending on the stimulation parameters used. This is thought to be attributable to both thermal effects (e.g., increasing tissue temperature decreases electrical impedance) and non-thermal effects (e.g., ultrasound may reduce the reactance portion of tissue impedance). Neural structures in particular have viscoelastic properties that are susceptible to electrical impedance variations caused by ultrasound waves. Low-frequency ultrasound has lower attenuation and higher intensities can be reached at deeper structures as compared with higher frequency ultrasound. As a result, low-frequency (e.g., less than 1 MHz) focused ultrasound can be used to reduce the electrical impedance of tissue at a treatment site, for example at or adjacent to a nerve. The reduction in electrical impedance can help “steer” externally applied electrical current to the treatment site, thereby improving the nerve modulation effects. In addition to ultrasound-based impedance effects, the electrical impedance of biological tissue may decrease with increasing electrical frequency. As a result, high frequency electrical currents (e.g., 50 kHz or more) may penetrate more deeply into tissue than lower frequency electrical currents, such as are used with conventional TENS treatment or interferential TENS therapy. Combining high-frequency electrical currents with focused ultrasound can lead to even greater reduction in tissue impedance at the treatment site.

Accordingly, the present technology relates to devices that combine TENS bipole electrode pairs with focused ultrasound (FUS) to increase current density at treatment site (e.g., a site or region positioned beneath a patient's skin). As described in more detail below, in some embodiments FUS can be applied to a treatment site within an acoustic frequency range of between about 20 kHz to 10 MHz concurrently with TENS bipole electrode pairs with an electric frequency range of between about 1 Hz to 100 MHz. Combining these modalities into a single device is intended to improve therapeutic outcomes by increasing the total amount of electric current delivered in addition to focusing the electrical current onto deep neurological structures of interest. Multiple TENS pads, for example including of 2-5 bipole electrode pairs, may be arranged in a circular or elliptical fashion to increase current flow at the midpoint of the TENS bipoles across different radial paths at a particular tissue depth. FUS can be used to increase the temperature of the area of interest to reduce focal electrical impedance, which will steer more current to be delivered to the area of interest from the surrounding tissue. FUS can also be used to directly activate or inactivate nerves via slight temperature increases or nonthermal effects independent of the application of TENS current. In some embodiments, additional electrical energy can be induced via one or more electromagnets (e.g., one or more electromagnetic coils) or other suitable technique, thereby further increasing the current density at the treatment site. In some embodiments, the device may be arranged in a puck configuration that can be adhered to the skin, and either one or two devices can be applied over a treatment site for bilateral coverage. Embodiments of the device may be used to treat pain and non-pain indications involving abnormal neural signaling in the central and/or peripheral nervous systems, including type 2 diabetes, urinary incontinence, Parkinsonian and essential tremor, autism, dementia, cancer, as well as inflammatory disorders including Rheumatoid arthritis, Crohn's disease, and ulcerative colitis.

In some embodiments, electrodes (e.g., bipole electrode pairs) can also provide superficial heating, which may be beneficial for nerve stimulation, nerve inhibition, and/or muscle stimulation application. For example, bipole electrode pairs for delivering electrical current can also be configured to generate heat (e.g., by running current through a resistive element adjacent to the user's skin). As such, the electrodes may deliver superficial therapeutic heat in addition to therapeutic electrical currents. This heat may be particularly beneficial for pain conditions and can operate analogously to TENS currents for pain, which act to stimulate superficial nerves to mask pain sensations actually occurring from deeper anatomic insults around the vertebral column. In some embodiments, the electrode pad may involve a removable gel pad for heat conduction and current coupling that goes over the electrode, may resemble a traditional TENS pad, or may involve metallic electrodes that require the treatment area to be slightly dampened prior to treatment.

In some embodiments, an ultrasound transducer (e.g., a focused ultrasound transducer) can be used independently to treat pain or other conditions without the use of electrical currents. For example, a focused ultrasound device having a focal zone of approximately 5 cm may be used to heat a zone deep below the surface of the skin, such as for heating the lumbar vertebral area. Such a transducer may be integrated into a handheld device, coupled to a wearable article (e.g., a lumbar belt, posture-support belts, etc.), or otherwise applied to the user's skin for delivery of targeted heat well below the surface of the user's skin.

### II. Example Noninvasive Neuromodulation Devices

### Neuromodulation Device Overview

In the illustrated embodiment, the neuromodulation device 100 comprises a controller 101 and a power source 103 (e.g., a battery or an electrical connection component for receiving facility power). The controller 101 can include, for example, a suitable processor or central processing unit (“CPU”) that controls operation of the device 100 in accordance with computer-readable instructions stored on the memory 105. The controller 101 may be any logic processing unit, such as one or more CPUs, digital signal processors (DSPs), application-specific integrated circuits (ASICs), etc. The controller 101 may be a single processing unit or multiple processing units in a device or distributed across multiple devices. The controller 101 is connected to the memory 105 and may be coupled to other hardware devices, for example, with the use of a bus (e.g., a PCI Express or Serial ATA bus). The memory 105 can include read-only memory (ROM) and random access memory (RAM) or other storage devices, such as disk drives or SSDs, that store the executable applications, test software, databases and other software required to, for example, implement the various routines described herein, control device components, communicate and exchange data and information with remote computers and other devices, etc.

The controller 101 also includes drive circuitry configured to control operation of modulation components 107 of the device 100. For example, the drive circuitry can be configured to deliver waveforms having predetermined and controllable parameters to one or more of the modulation components 107 which can include one or more electrode(s) 109 (e.g., transcutaneous electrical nerve stimulation (TENS) electrodes or other suitable electrodes), one or more focused ultrasound (FUS) transducers 111, and/or one or more electromagnetic (EM) coils 113. In some embodiments, the device 101 omits one or more of these modulation components 107, for example including only electrodes 109 and FUS transducers 111, while omitting EM coils 113, or including only electrodes 109 and EM coils 113 while omitting FUS transducers 111, etc.

As described in more detail below, in some embodiments the electrodes 109 include a plurality of bipole electrode pairs, or in some embodiments only a single bipole electrode pair. These bipole electrode pairs may be arranged around an X-shaped, circular, elliptical, or other pattern in which each of the electrode pairs is substantially aligned along a plane and configured to contact a patient's skin. For example, a first bipole electrode pair may be aligned along a first axis and configured to contact a patient's skin, and a second bipole electrode pair may be aligned along a second axis and also configured to contact the patient's skin, such that the first and second axes intersect at a point on the patient's skin over the treatment site.

In operation, bipole electrode pairs 109 can transmit current from one electrode to the other along a curved path through the skin, in a distribution pattern resembling a canoe or banana. These curved paths intersect for different bipole pairs, and can combine together at a treatment site below the surface of the skin. The FUS transducer 111 can be configured to transmit focused ultrasound along an axis substantially orthogonal to the patient's skin, penetrating to a desired depth. The FUS can lower the impedance of tissue at the focal zone due to thermal and non-thermal effects, thereby steering current into that region of tissue. The combination of overlapping current flows from different bipole electrode pairs and the impedance-lowering effects of the FUS beam cause electrical current to concentrate at a treatment site at a desired depth below the surface. In some embodiments, alternating current applied to the EM coil 113 can also be used concurrently with the electrodes 109 and the FUS transducer 111 to provide additional electrical current directed towards the treatment site. The treatment site can be a target nerve of a patient such that concentration of current at the target nerve provides therapeutic benefits, such as pain relief, reduction of sympathetic hyperactivity, or other clinically beneficial effects.

As noted elsewhere herein, in some embodiments the bipole electrode pairs 109 can themselves deliver superficial heat to the treatment site. For example, running current through one or more resistive elements adjacent to the user's skin may generate heat in those elements that is conducted to the skin, either through direct contact or through an intervening thermal conductor such as a gel, pad, or paste. In some embodiments, this superficial heat may stimulate superficial nerves, for example to mask pain sensations actually occurring from deeper anatomic insults around the vertebral column.

The controller 101 can provide information and instructions to device users via the display 115 or other suitable output device (e.g., audible output via speakers, tactile output, etc.). The controller 101 can also receive user inputs via input 117, which can take the form of a touch screen, dials, knobs, switches, buttons, keys, microphones, cameras, or other suitable input elements. In some embodiments, the controller 101 may mediate the power provided from power source 103 to the modulation components 107 based on the user inputs, for example varying the waveforms, amplitude, duration, duty cycle, frequency, or any other aspect of the modulation components 107. The inputs may also toggle on/off different components of the device 100, for example separately turning on/off the electrodes 109, the FUS transducers 111, and/or the EM coils 113.

In some embodiments, the controller 101 can permit a user to select between different modes of operation, for example a first frequency (or range of frequencies) for electrical muscle stimulation, a second frequency (or range of frequencies) for electrical nerve stimulation, and a third frequency (or range of frequencies) for electrical nerve inhibition. The user may also be able to toggle between a superficial heating mode and a non-heating mode, as well as toggling operation of the FUS transducer 111 or EM coils 113 (if applicable).

The device 100 may also include a communication link 119, which can include a wired connection (e.g., an Ethernet port, cable modem, FireWire cable, Lightning connector, USB port, etc.) or a wireless connection (e.g., including a Wi-Fi access point, Bluetooth transceiver, near-field communication (NFC) device, and/or wireless modem or cellular radio utilizing GSM, CDMA, 3G and/or 4G technologies) for data communication with all manner of remote processing devices via a network connection and/or directly via, e.g., a wireless peer-to-peer connection. For example, the communication link 119 can facilitate wireless communication with handheld devices, such as a mobile device 123 (e.g., a smartphone, blood glucose monitor, etc.) either in the proximity of the device 100 or remote therefrom.

In some embodiments, the neuromodulation device 100 includes one or more temperature sensors 121. Such a temperature sensor 121 can be configured to obtain temperature measurements from a patient's skin while the device 100 is in use. Alternatively or additionally, the temperature sensor 121 can be configured to obtain temperature readings at a site or region beneath a patient's skin. In some embodiments, the controller 101 can be coupled to the temperature sensor(s) 121 and configured to turn off or reduce power of the modulation components 107 if the temperature sensor(s) 121 detect a temperature above a predetermined threshold (e.g., an increase of more than 5° F., more than 10° F., etc.). This can reduce or eliminate the risk of skin burns or other injury due to excess heating caused by the modulation components 107. As noted elsewhere herein, in some instances superficial heating may be desired, and may be achieved via the bipole electrode pairs 109 or other separate heating elements. In such embodiments, the temperature sensor(s) can monitor this superficial heating to ensure that efficacious heating is achieved without risking burning or other damage from excessive superficial heating.

As noted above, in some embodiments, the neuromodulation device 100 may also communicate with a mobile device 123. The mobile device 123 can include one or more features, applications and/or other elements commonly found in smartphones and other known mobile devices. For example, the mobile device 123 can include a processor 125 (e.g., a CPU and/or a GPU) for executing computer readable instructions stored on memory 127. In addition, the mobile device 123 can include an internal power source 129 such as a battery, and well-known input components 131 and output components 133, including, for example, a touch screen, a keypad, speakers, a camera, etc. In addition to the foregoing features, the mobile device 123 can include a communication link 135 (e.g., a wireless transceiver that may include one or more antennas for wirelessly communicating with, for example, other mobile devices, websites, and the neuromodulation device 100. Such communication can be performed via, e.g., a network (which can include the Internet, public and private intranet, a local or extended WiFi network, cell towers, the plain old telephone system (POTS), etc.), direct wireless communication, etc. In various embodiments, the mobile device 123 can be a smartphone, blood glucose monitor, other portable physiological sensor, fitness monitor, smart watch, or any other suitable device.

In some embodiments the device 100 may be controlled at least in part based on instructions or other input received from the mobile device 123. For example, the mobile device 123 may include a mobile application (or “app”) that allows a user to modify therapy delivered via the modulation components 107 including, for example, duration, amplitude, waveform, frequency, duty cycle, or any other parameter. In some embodiments, the mobile device 123 may be configured to monitor other physiological parameters, for example blood glucose levels, heart rate, blood pressure, etc. Based on measurements of physiological parameters obtained via the mobile device 123, the neuromodulation device 100 may vary application of the modulation components 107.

### Electrode Components

As noted previously, the modulation components 107 include electrodes 109, which can take the form of TENS electrodes. These electrodes can include conductive pads with an adhesive portion such that they can be adhered to a user's skin. An electrical lead extending from the conductive pads can be electrically coupled to the controller 101, which drives current through the electrodes 109. In at least some embodiments, the electrodes 109 include a plurality of bipole electrode pairs, for example two, three, four, five, or more bipole electrode pairs. These pairs can be arranged such that current primarily travels between one electrode of the bipole pair to the other. In some embodiments, the electrodes can be arranged as component sections of puck, disc, or other similar arrangement with each section electrically isolated from adjacent sections.

In operation, these electrode bipole pairs can be arranged along different axes (e.g., with each bipole electrode pair defining an axis) that intersect one another. The electrodes of each pair can be separated from one another along the respective axis. In some embodiments, each electrode is separated from its pair electrode by about 5-15 cm. In some embodiments, each electrode is separated from its pair electrode by more than 5 cm, more than 6 cm, more than 7 cm, more than 8 cm, more than 9 cm, more than 10 cm, more than 11 cm, more than 12 cm, more than 13 cm, more than 14 cm, or more than 15 cm. These pairs may be arranged in a circular or elliptical fashion to increase current flow at the midpoint of the bipoles across different radial paths. In operation, some or all of these electrodes 109 can be adhered to or otherwise in contact with a patient's skin. Electrical energy applied to the electrodes 109 (e.g., via the controller 101) causes current to flow between the individual pairs along various depths within the tissue. At or near the intersection of the different axes along which the bipole pairs are arranged, the lines of current can intersect, creating a site or region of increased current density.

In various embodiments, the electrical energy applied to the electrodes 109 can be alternating current in the range of about 1 Hz to 100 MHz, for example between about 1-300 kHz, etc. In some embodiments, the electrodes 109 can be driven with AC of more than 1 kHz, more than 50 kHz, more than 100 kHz, more than 150 kHz, more than 200 kHz, more than 250 kHz, or more than 300 kHz. The electrodes 109 can be driven with current in the range of 0 to about 500 mA, for example 10 to 100 mA. The AC can be applied using a variety of different waveforms, duty cycles (e.g., a duty cycle ranging anywhere from 0-100, etc. In some embodiments, the bipole pairs of the electrodes 109 can be fired simultaneously and with the same frequency such that the bipole pairs are in phase with one another. In some embodiments, the electrodes 109 can be driven with a waveform characterized by a pulse repetition frequency, for example between about 0-1 kHz. In some embodiments, a device configured for nerve inhibition can have a pulse repetition frequency of around 100 Hz plus or minus 50 Hz, or for example between about 30 to 200 Hz, or between about 50 and 150 Hz, or between about 60 and 109 Hz. In some embodiments, the waveform can include a burst frequency (e.g., the pulse repetition frequency) and an intra-burst frequency (e.g., the underlying waveform frequency). The waveform driving the electrodes 109 can also vary in shape, for example a sine wave, square wave, triangular wave, sawtooth, any combination of these shapes, or any other suitable shape.

In some embodiments, the waveform driving the electrodes 109 can be a square wave, having positive (anodic) and negative (cathodic) portions of the wave. Such waveforms can be characterized by a pulse width, for example having a pulse width ranging from about 1 nanosecond to about 1 second. The pulse width may refer to either the anodic width or cathodic width, which in some embodiments may be identical to ensure charge balance. In some embodiments, the waveform may also be characterized by interphase delay, reflecting the time delay between the positive and negative portions of the wave. In various embodiments, the interphase delay for a square wave or any other waveform may be between about 1 nanosecond and about 1 second. In some embodiments, there is no interphase delay. In one example, a square wave having a 5-nanosecond pulse width for each of the positive and negative portions of the waveform, with no interphase delay, corresponds to a 100 MHz wave, while a square wave with a 4-nanosecond pulse width and a 1 nanosecond interphase delay after each positive and negative portion would also correspond to a 100 MHz wave. In both cases, the total phase time of 10 nanoseconds (two 5-nanosecond pulses in the former case; two 4-second nanosecond pulses plus two 1-nanosecond interphase delays in the latter) corresponds to a frequency of 100 MHz.

### Focused Ultrasound Components

The modulation components 107 can also include one or more focused ultrasound (FUS) transducers 111. Although many examples below describe a single FUS transducer 111, in various embodiments there may be two, three, four, or more FUS transducers 111. In some embodiments, the FUS transducer 111 includes a spherical or cylindrical FUS transducer configured to direct ultrasound energy towards a treatment site beneath a patient's skin. For example, with the electrodes 109 arranged over a patient's skin as described previously, the FUS transducer 111 can be arranged to deliver ultrasound energy along an axis substantially orthogonal to a patient's skin (e.g., substantially orthogonal or at least intersecting with a plane along which two or more of the electrodes 109 are aligned). In some embodiments, the FUS beam can be configured to intersect with an intersection point of the bipole electrode pairs described above.

To establish ultrasound coupling with a patient's skin, the FUS transducer 111 can be configured to be placed into contact with the patient's skin during operation of the device 100. In some embodiments, a gel pad or other similar ultrasonically conducting material can be used to couple the FUS transducer 111 to the patient's skin.

In some embodiments, the FUS transducer 111 can be a spherical or cylindrical transducer having a radius of curvature (ROC) between 5-500 mm, for example between 15-100 mm, or in some embodiments about 60 mm. In some embodiments, the FUS transducer 111 can include a lens or lens array that focuses the ultrasound beam to a desired focal zone. In some embodiments, the FUS transducers 111 can be characterized by an inner diameter (e.g., the diameter of an aperture on the rear surface of the FUS transducer 111), for example having an inner diameter of between about 0-50 mm, or in some embodiments about 43 mm.

Depending on the configuration of the FUS transducer 111, the focal length and/or the size of the focal zone can be varied to achieve a desired ultrasound beam. There are several working definitions of a focal zone, for example, 3 dB, 6 dB, and full-width-at-half-maximum (FWHM) acoustic pressure field focal regions. The focal length can be selected to correspond to particular tissue depths and particular neurological structures (e.g., a treatment site or region). In some embodiments, the focal length can be between about 25-90 mm FWHM so as to target nerve structures positioned within this range beneath a patient's skin.

As noted above, the focal zone of the FUS transducer 111 can establish the point of highest current density at the treatment site, e.g., by heating the treatment site and thereby decreasing its electrical impedance. Accordingly, it can be desirable to provide a focal zone having dimensions that correspond to particular anatomical structures, for example a treatment zone that is largely or completely contained within the volume of a target nerve. In one particular example, the focal zone can be selected to have a width and depth corresponding to a width and depth of a dorsal root ganglion. In some embodiments, the width of the focal zone is between about 5-15 mm. In some embodiments, the depth of the focal zone is between about 2-10 mm. However, in various embodiments, the focal length and/or the dimensions of the focal zone can vary in order to better target particular treatment sites beneath a patient's skin.

In various embodiments, the FUS transducers 111 can be driven to output ultrasound energy having a frequency in the range of about 100 kHz to about 10 MHz, or about 250-2000 kHz, or about 1 MHz. The ultrasound energy can be emitted according to a variety of different waveforms, duty cycles, etc. In general, higher frequencies of ultrasound can be focused more precisely into smaller focal zones, allowing for more precision targeting of treatment sites. However, to modulate certain target nerves, it can be advantageous to have a relatively wider focal zone (e.g., a focal zone having a width of approximately 5-10 mm wide allows for steering current to a focal zone that corresponds to a width of the dorsal root ganglion).

In various embodiments, the FUS transducers 111 can be driven with pulsed DC energy, AC energy, or any other suitable driving current. The drive current can be approximately 10-2000 mA of current, driving intensities around 0.1-10 W/cm², corresponding to around 0.1-10° C. temperature increases in and around the spinal cord. In various embodiments, the FUS transducer(s) can deliver ultrasound to the treatment site at an intensity of between about 0.1 and about 10,000 W/cm2, for example between about 0.1-10 W/cm².

In some embodiments, a waveform driving the FUS transducer(s) 111 can be characterized by a duty cycle between 1-100%, for example about 30%, about 40%, about 50%, etc. The driving waveform may also be characterized by a pulse repetition frequency of any value between 0-10 kHz. The pulse repetition frequency corresponds to the frequency (1/period) of distinct ultrasound wave pulses, while the duty cycle indicates the percentage during each ultrasound wave pulse that the ultrasound is in the ON state. For example, a 500 kHz ultrasound signal, with a pulse repetition period of 2 ms, has a PRF of 500 Hz (1/0.002). A 50% duty cycle indicates 1 ms of 500 kHz ultrasound ON, followed by 1 ms of no ultrasound energy, in a repeating cycle. As with the waveform driving the electrodes 109, the waveform driving the FUS transducer(s) 111 can assume a variety of shapes, for example a sine wave, square wave, triangular wave, sawtooth, any combination of these shapes, or any other suitable shape.

### Electromagnetic Coil Components

In some embodiments, the device 100 includes one or more EM coils 113 that can provide additional or alternative electrical energy to a treatment site. Similar to transcranial magnetic stimulation (TMS) coils, the EM coil 113 may be driven with electrical current, thereby inducing a magnetic field along an axis substantially orthogonal to the plane of the EM coil 113. This magnetic field, in turn, induces a secondary electric field arranged in a circular fashion at a tissue depth below the surface. In some embodiments, the EM coil 113 may be arranged such that the induced secondary electrical current beneath the surface is substantially aligned with the focal zone of the FUS transducer(s) 111 and/or substantially aligned with the intersection point of the bipole electrode pairs 109. In some embodiments, the EM coils 113 can induce an electrical current in the tissue having a similar or identical frequency to that provided by the electrodes 109, such that the electrical current in the tissue from the two sources is cumulative at the treatment site. In other embodiments, the EM coils 113 can induce an electrical current in the tissue having markedly different properties, for example being significantly higher frequency than that of the electrodes 109.

In various embodiments, the EM coils 113 can be driven with electrical energy having a current of between about 1 mA and about 100 kA. The driving current of the EM coils 113 can have a frequency of between about 1 kHz to about 10 GHz, for example between about 100 kHz and 100 MHz.

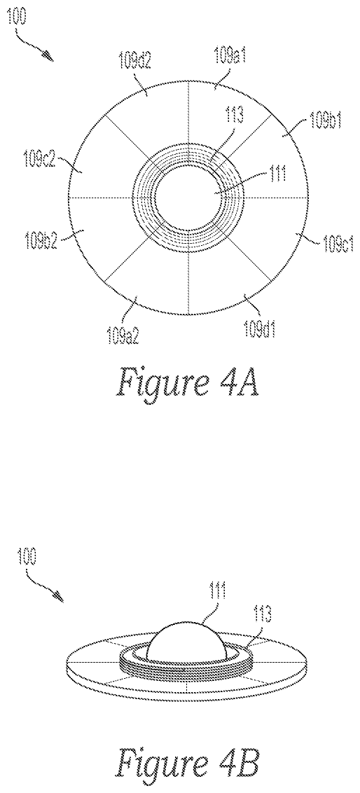

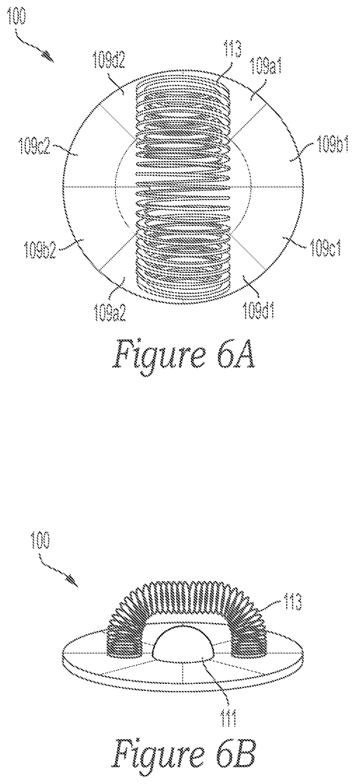

The EM coils 113 may take a variety of forms, for example wrapped wire made of copper or other conductive metal, other materials such as graphene, conductive ceramics, or any other suitable electrically conductive material. The coil may be a continuous strand of single wire, or a multi-wire construction such as Litz wire. The coil may be compressed into a generally cylindrical shape, or may be separated into a “slinky” configuration as shown in FIGS. 6A and 6B or any other suitable shape. The arrangement, position, and configuration of the EM coils 113 can vary, as described in more detail below with respect to FIGS. 4A-6B.

In embodiments in which the EM coils 113 induce an electrical current having a relatively low frequency (e.g., ˜100 kHz), it may be necessary to use high driving voltage and/or relatively bulky superconducting coils, for example coils made from niobium titanium wires that are helium-cooled and embedded in copper billets, similar to MM machines. This may be useful when the EM coils 113 are used to induce an electrical current at the treatment site having a frequency that substantially corresponds to a frequency of the electrodes 109.

However, in embodiments in which the EM coils 113 are used to induce a higher frequency current at or near the treatment site, non-superconducting coils (e.g., coils made of copper or other suitable material) may be used to generate electrical current in the MHz range. Because higher frequency current may generate “skin” effects that increase AC resistance in the coil, it can be useful to use thinner wires that are less susceptible to such skin effects. For example, using a copper wire with a higher number of turns with high electrical current can allow the EM coil 113 to produce current at the treatment site having MHz frequencies. These induced MHz currents will generally be non-localized and will spread over a larger region than lower frequency current, due in part to the lower electrical impedance of biological tissue at MHz frequencies. As such, these higher frequency and more highly dispersed currents may be suitable for applications in which a larger area is targeted. One example is treatment of fibromyalgia, which is a widespread pain disorder. For example, a single device applied to either the back of the neck or the lower back may provide therapeutic effects to both local and more distant tender points associated with fibromyalgia. Since the current is induced using an EM coil and is not constrained between two bipoles, there can be a more widespread current path as compared to conventional TENS therapy.

In some embodiments, the EM coil(s) 113 may be used without the FUS transducers 111 and/or without the electrodes 109 for treatment of fibromyalgia or similar disorders. For example, the EM coil(s) 113 may be driven with a current sufficient to induce an electrical current in the tissue of 150 uA or more, having a frequency of between about 400 kHz to about 5 MHz. This high frequency current may spread significantly from the position of the EM coils 113, thereby potentially modulating nerves within a relatively broad treatment site.

### Controller and Drive Circuitry Components

As noted previously, the controller 101 can be configured to control operation of the device 100 and, in particular, to control operation of modulation components 107. In some embodiments, the controller 101 comprises a suitable processor or central processing unit (“CPU”) that controls operation of the device 100 in accordance with computer-readable instructions stored on the memory 105. The controller 101 may be any logic processing unit, such as one or more CPUs, digital signal processors (DSPs), application-specific integrated circuits (ASICs), etc. The controller 101 may be a single processing unit or multiple processing units in a device or distributed across multiple devices.

The controller 101 also includes drive circuitry configured to control operation of modulation components 107. For example, the drive circuitry can control various parameters of the energy output by the power source 103 to the modulation components 107, such as intensity, amplitude, duration, frequency, duty cycle, and polarity. In some embodiments, controller 101 includes one or more current generators, amplifiers, transformers, or any other suitable circuit elements to drive the electrodes 109, FUS transducer 111, and/or the EM coil 113 in addition to other elements of the neuromodulation device 100. In various embodiments, the controller 101 can include hardwired circuit elements to provide the desired waveform delivery rather than a software-based current generator. The drive circuitry can include, for example, analog circuit elements (e.g., resistors, diodes, switches, etc.) that are configured to cause the power source 103 to deliver electric to one or more of the modulation components 107 according to the desired parameters. In some embodiments, the controller 101 can include separate drive circuitry for each of the modulation components 107—e.g., first drive circuitry for the electrodes 109, second drive circuitry for the FUS transducer 111, and third drive circuitry for the EM coils 113.

In some embodiments, the various modulation components 107 may be driven at varying frequencies. For example, the electrodes 107 may be driven with AC having a frequency of between about 1 kHz to about 1 MHz, for example between about 1-300 kHz, or between about 20-100 kHz. Meanwhile, the EM coil 113 can be driven with AC having a frequency of between about 1 kHz to about 10 GHz. In some embodiments, the FUS transducer 111 can be driven with electric current to output an ultrasound frequency of between about 100 kHz to about 10 MHz, for example between about 250-750 kHz.

### Example Device Arrangements

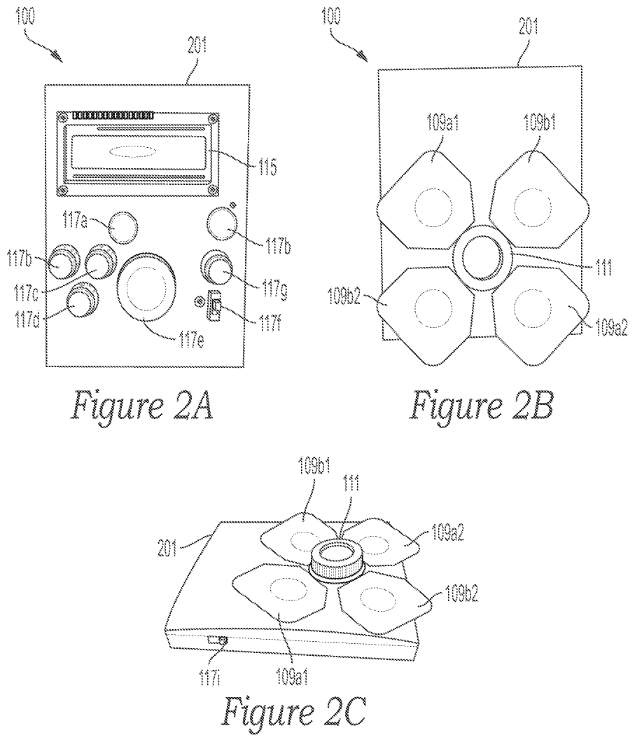

FIGS. 2A-2C are front, rear, and rear perspective views, respectively, of a neuromodulation device. The device 100 shown in FIGS. 2A-2C includes a housing 201 having an upper surface on which an output display 115 and a variety of input elements 117a-g) are disposed. The modulation components (e.g., the electrodes 109 and the focused ultrasound transducer 111) are disposed over a lower surface of the housing 201. The housing 201 can encompass a controller, power source, and any other electronic components (not shown). The housing 201 can be generally rectangular, for example having a height or thickness of approximately 3-5 cm, a width of approximately 10-15 cm, and a length of approximately 15-20 cm. However, other dimensions are possible in various embodiments. The housing 301 and the device 100 can assume other shapes, configurations, and dimensions (both smaller and larger) depending on the particular components included, the desired treatment site, power requirements, and other parameters.

The input elements 117a-g include a variety of knobs, buttons, and switches that can be used to turn the device on or off, set treatment times (e.g., activate the device for a predetermined time period, after which the device will automatically turn off), adjust the frequency, current, waveform, and/or other parameters controlling the electrodes 109 and the FUS transducer 111.

As illustrated, the electrodes 109 include two electrode bipole pairs: 109a1-a2 and 109b1-b2. In operation, the device 100 can be placed over a patient's skin adjacent to a treatment site such that the electrodes 109 and the transducer 111 are all in contact with the patient's skin. Once in this position, current flows between each pair (e.g., between 109a1 and 109a2, and between 109b1 and 109b2), such that the current paths across each of these two bipole electrode pairs can intersect at a central region that is substantially aligned with the FUS transducer 111. At least some of the current passing between individual electrodes of a bipole pair may pass transcutaneously at a depth below a patient's skin when the device 100 is placed over a treatment site. To increase current penetration depth, it can be beneficial to maximize the surface area of the electrodes 109 further away from a center region and to minimize the surface area towards the center region, as the center region may tend to attract current more superficially. Accordingly, the individual electrodes 109 can have a tapering shape, with a wider portion at a distance further from a central region, and a narrower portion at a distance closer to the central region in which the FUS transducer 111 is positioned.

The FUS transducer 111 can be a spherical transducer oriented to direct a focused ultrasound beam along an axis generally orthogonal to the plane along which the electrodes 109 are arranged. This allows the FUS transducer 111 to directed ultrasound energy to a treatment site, thereby steering electrical current provided by the electrodes 109 towards the treatment site, as discussed in more detail below.

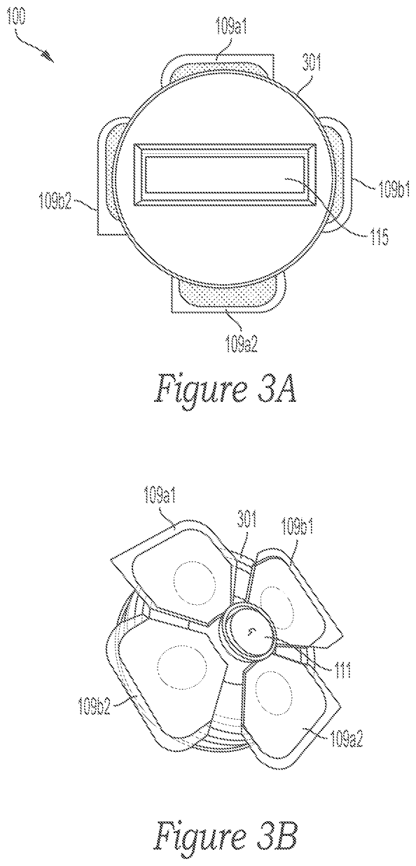

FIGS. 3A and 3B are front and rear perspective views, respectively, of another neuromodulation device 100. The embodiment illustrated in FIGS. 3A and 3B can be similar to that described above with respect to FIGS. 2A and 2C, except that a circular housing 301 is provided that can encompass a controller, power source, and any other electronic components (not shown). A display screen 115 is disposed over an upper surface of the housing 301. Additional control input and output can be provided on the housing 301, for example around the perimeter, via touch input, or via other control mechanisms. The modulation components (the electrodes 109 and the FUS transducer 111) are disposed over a lower surface of the housing 301. The housing 301 can be a generally cylindrical puck, for example having a height of approximately 3-5 cm and a diameter of approximately 10-15 cm. However, other dimensions are possible. The housing 301 and the device 100 can assume other shapes, configurations, and dimensions (both smaller and larger) depending on the particular components included, the desired treatment site, power requirements, and other parameters. As illustrated, the electrodes 109 include two electrode bipole pairs: 109a1-a2 and 109b1-b2. In operation, current flows between each pair (e.g., between 109a1 and 109a2, and between 109b1 and 109b2), such that the current paths across each of these two bipole electrode pairs can intersect at a central region that is substantially aligned with the FUS transducer 111.

FIGS. 4A and 4B are top and side perspective views, respectively, of another neuromodulation device. The illustrated embodiment 100 includes eight separate electrodes arranged in four electrode bipole pairs: 109a1-a2, 109b1-b2, 109c1-c2, and 109d1-d2 (collectively “electrodes 109”). In operation, current flows between each pair (e.g., between 109a1 and 109a2), such that the current paths across each of these four bipole electrode pairs can intersect at a central region. The electrodes 109 can be arranged in generally circular shape, with electrodes being disposed adjacent to one another with an opening in a central region that is not conductive. This allows current flowing from one electrode bipole to the other to pass subcutaneously rather than pass only superficially across the device 100. In the illustrated embodiment, the electrodes 109 are arranged in a pizza-slice configuration, with an opening in the central region. In other embodiments, the electrodes 109 can take other forms, for example with each being physically separated from one another rather than being unified in a puck-like layer or housing. Similarly, although a circular arrangement is illustrated here, in other embodiments the electrodes can be arranged in a square, rectangular, elliptical, or other fashion.

With continued reference to FIGS. 4A and 4B, the focused ultrasound transducer 111 is disposed in the central region between the arrayed electrodes 109. The FUS transducer 111 can be a spherical transducer oriented to direct a focused ultrasound beam along an axis generally orthogonal to the plane along which the electrodes 109 are arranged. This allows the FUS transducer 111 to directed ultrasound energy to a treatment site, thereby steering electrical current provided by the electrodes 109 towards the treatment site, as discussed in more detail below.

An EM coil 113 is disposed circumferentially around the FUS transducer 111. For example, the EM coil 113 can be a conductive wire wrapped cylindrically. In operation, alternating current applied to the EM coil 113 can induce a changing magnetic field oriented along an axis orthogonal to the plane of the electrodes 109 (e.g., along an axis parallel to the axis of the ultrasound beam emitted by the FUS transducer 111). This changing magnetic field in turn induces a secondary electrical field at a position spaced apart from the EM coil 113, for example at a treatment site beneath a patient's skin. This secondary electrical field provided by the EM coil 113 can contribute additional electrical energy to be combined with the electrodes 109 and the steering effects of the focused ultrasound to provide modulation to one or more nerves at a treatment site.

Each of the electrodes 109, the FUS transducer 111, and the EM coil 113 can be electrically coupled to a controller (not shown) that includes a power source, drive electronics, etc. configured to control operation of these various components.

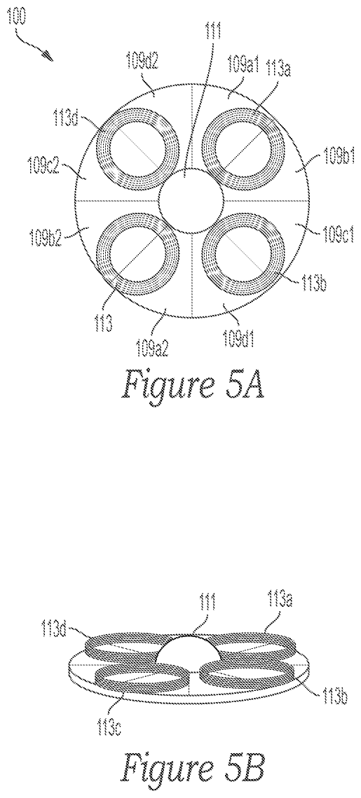

FIGS. 5A and 5B are top and side perspective views, respectively, of another neuromodulation device. The device 100 illustrated in FIGS. 5A and 5B can be similar to the embodiment illustrated in FIGS. 4A and 4B, except that in place of the single EM coil 113 surrounding the FUS transducer 111, the embodiment of the neuromodulation device 100 shown in FIGS. 5A and 5B includes four EM coils 113a-d disposed over the surface of the electrodes 109 and laterally spaced apart from the FUS transducer 111. In operation, each of these EM coils 113 can induce a circular electrical field below the surface of the skin, which can increase the total current supplied to the treatment site when combined with electrical current supplied by the electrodes 109. Although the illustrated embodiment has four EM coils 113a-d, in various embodiments there may be one, two, three, five, six, or more EM coils 113 provided at any number of different positions and orientations with respect to the FUS transducer 111 and/or the electrodes 109.

FIGS. 6A and 6B are top and side perspective views, respectively, of another neuromodulation device. The device 100 illustrated in FIGS. 6A and 6B can be similar to those embodiments illustrated in FIGS. 4A-5B, except that here the EM coil 113 takes the form of an extended coil that spans over the FUS transducer 111, with each end of the EM coil 113 adjacent to the electrodes 109 at a different position, taking the form of a slinky-like arrangement. Although a single extended EM coil 113 is illustrated here, in other embodiments two, three, or more similar EM coils 113 can be provided, each spanning over the FUS transducer 111 along a different axis. In at least some embodiments, the extended EM coil 113 can result in increased electrical current at the treatment site as compared to a collapsed arrangement in which the EM coil 113 is stacked cylindrically. Without wishing to be bound by theory, such an extended EM coil 113 may operate analogous to a horseshoe magnet, with more current being induced because both poles of the magnet are against the skin.

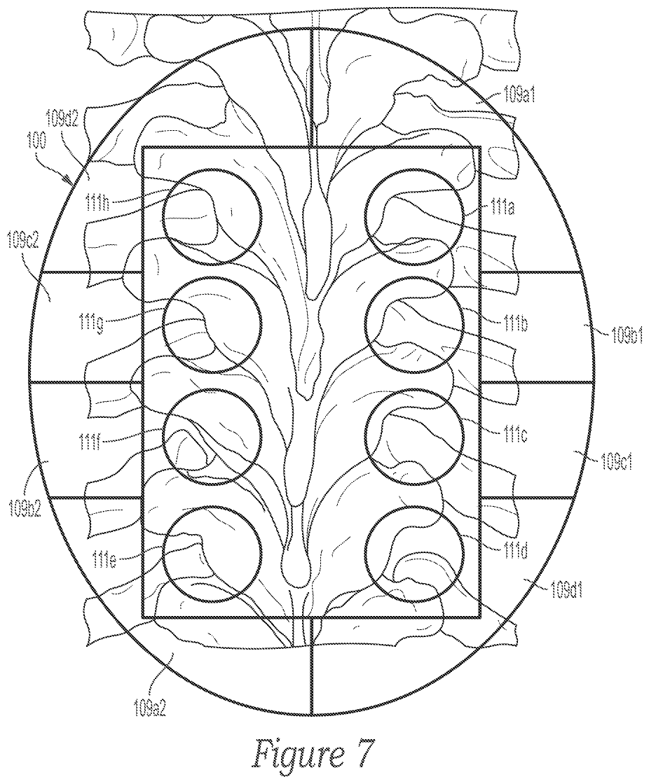

FIG. 7 is a top view of another neuromodulation device positioned over a spine of a patient. In the illustrated embodiment, 4 pairs of FUS transducers 111a-h are arranged on opposing sides of the vertebral column. Each spinal level may have two FUS transducers 111, one on either side of the vertebral column, and each pair may be vertically situated approximately in between the transverse processes of the vertebrae above and below the pair of FUS transducers 111. In this arrangement, each focused ultrasound beam may target an area in between two adjacent transverse processes. In various embodiments, there may be 2-8 pairs of FUS transducers 111, which can be arranged to span a region of the spine, for example the T5-T12 region or any region therebetween.

In addition to the FUS transducers 111, there is an associated electrode (e.g., 109a1) adjacent to each FUS transducer 111. For example, each pair of FUS transducers 111 may have an associated electrode bipolar pair with one electrode 109a1 to the left of the left FUS transducer 111, and another electrode 109a2 to the right of the right FUS transducer 111, so that the FUS pair lies in between the TENS bipole electrodes. There may also be additional TENS bipoles oriented in a cranial-caudal orientation on either side of the spinal cord, so that these pairs are situated perpendicular to the other TENS bipoles. The cranial-caudal TENS bipoles may have one its electrodes above the FUS transducer pairs and the other below. All of the FUS pairs and TENS bipoles will be encased in an oval pad that the patient places on his or her back over the T5-T12 vertebrae or other suitable region of the spine.

In another similar permutation, instead of each FUS pair having a TENS bipole, there could be two total TENS bipoles. One bipole would have one larger electrode spanning T5-T12 to the left of the spinal cord and to the left of the FUS pairs, and the other larger electrode would be the same size to the right of the spinal cord and to the right of the FUS pairs. The second bipole may be oriented in a cranial-caudal orientation and would have one larger electrode above the FUS pairs that crosses the midline and spans horizontally the at least the area of the FUS pair, and the other larger electrode of the same size may be below the FUS pairs and also cross the midline.

A device as shown in FIG. 7 may be particularly useful for treating type 2 diabetes. For treatment of type 2 diabetes, there are various sympathetic nerves that are anterior to the dorsal root ganglion that would be accessible for a FUS beam, such as the sympathetic trunk, white ramus communicans, the gray ramus communicans, or the greater splanchnic nerve. If the greater splanchnic nerve is not targeted directly, the greater splanchnic nerve fibers can be targeted as they each run through the white ramus communicans. The device of FIG. 7 allows for a broad, bilateral sympathetic inhibition. The device of FIG. 7 also permits a more fine-tuned control of modulation of various spinal levels that correspond to different organs, for example targeting T5 and T6 for hepatic sympathetic inhibition around meal times, and targeting T8 and T9 other times for adrenal gland sympathetic inhibition.

In operation, when both the FUS transducers 111 and the electrodes 109 are turned on, current can be steered to regions aligned with the respective focal zones of the FUS transducers 111. However, when the FUS transducers 111 are off but the electrodes 109 are turned on, the subcutaneous current likely spreads further anteriorly towards belly, where it may stimulate vagus nerves (which are anterior to splanchnic nerves). High frequency vagus nerve block has been used to treat obesity. Losing weight can be very useful for type 2 diabetes patients. Accordingly, the device 100 may periodically be used to delivery electrical current only (e.g., via the electrodes 109 without application of ultrasound via the FUS transducers 111). This may result in a less focused and effective splanchnic block, but may simultaneously provide a vagus block, thereby curbing appetite. This may be useful outside of mealtimes, while during mealtimes the device 100 may deliver both FUS and electrical energy, thereby providing a more effective splanchnic block.

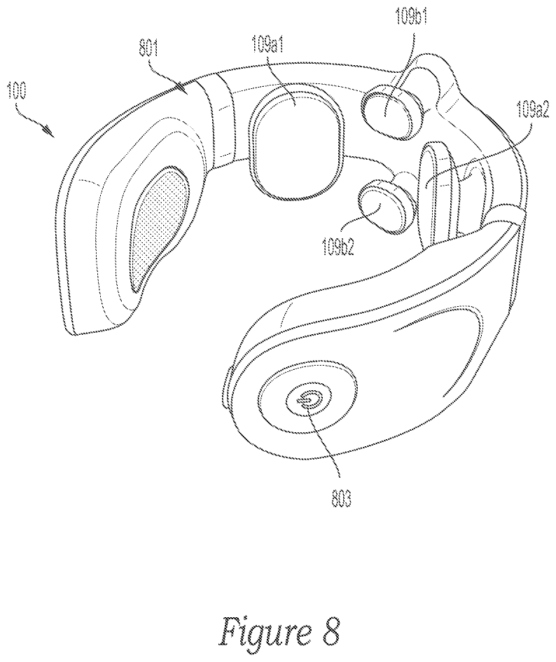

FIG. 8 illustrates another example neuromodulation device 100. In this embodiment, the device 100 is adapted for use in treatment of migraines, for example including a housing 800 configured to be placed around a user's neck. The device 100 includes four electrodes forming two bipole pairs (e.g., electrodes 109a1 and 109a2 form a first bipole pair and electrodes 109b1 and 109b2 form a second bipole pair). In operation, the device 100 can be disposed over a user's neck such that the bipole pairs are in contact with and facing the rear surface of the user's neck. In some embodiments, the electrodes can include metallic or other conductive surfaces, and in operation the user may apply water, conductive gel, or another suitable conductive material either to the surfaces of the electrodes or to the user's neck).

The user may initiate delivery of therapeutic current via the controller input 803, which can be a remote device (e.g., a smartphone or separate remote control) or a user interface (e.g., button, switch, etc.) integrated into the housing 801. The two bipole pairs arranged along orthogonal axes can each deliver current configured to penetrate the skin and inhibit nerves at the treatment site. In some embodiments, the electrodes can be configured to provide superficial heating as well. In some embodiments, a FUS transducer 111 can also be coupled to the housing 801 and configured to emit ultrasound towards the treatment site. Any suitable electrical currents can be used. In some embodiments, the two bipole pairs can each be driven with current of between about 500 Hz to about 5 MHz, for example around 1 MHz. Although the illustrated embodiment includes two perpendicular bipole pairs, in other embodiments a single bipole pair can be used.

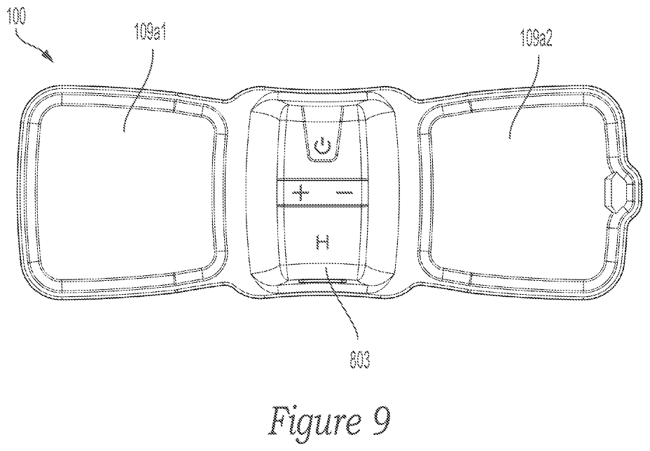

FIG. 9 illustrates another example neuromodulation device 100. In the illustrated embodiment, the device 100 takes the form of an elongated band that includes a single bipole electrode pair (e.g., electrode 109a1 and 109a2). As illustrated, the two electrodes 109a1-a2 are separated from one another across a length of the device 100. In operation, the device 100 can be placed over the treatment site (e.g., spanning over a user's back), with the treatment site being located underneath (or otherwise aligned with) the central region between the two electrodes 109a1-a2. The electrodes 109a1-a2 can include a gel pad to facilitate adhering the electrodes 109a1-a2 to the user's skin and to improve electrical conductivity between the electrodes 109a1a-a2 and the user's skin. In some embodiments, the device 100 is configured to apply superficial heating to the user's skin. For example, the electrodes 109a1-a2 can be configured to generate heat during operation, and that heat can be conducted to the skin over the treatment site (e.g., conducted through gel pads of the electrodes 109a1-a2).

In the central region between the electrodes 109a1-a2 is a controller input 803, which permits a user to toggle the device on/off as well as to increase or decrease the intensity of the current delivered via the electrodes 109a1-a2. The controller input 803 also permits a user to toggle on/off the application of superficial heat. For example, a resistive element disposed in one or both of the electrodes 109a1-a2 can be used to generate heat (e.g., by delivering current through the resistive element). In some embodiments, this heating can be controlled independently of the electrical current delivered via the electrodes 109a1-a2. In other embodiments, the heating and the electrical current can be applied and controlled concurrently. In some embodiments, the controller input 803 can permit a user to toggle between modes having different frequencies or frequency ranges. For example, a first frequency (or range of frequencies) for electrical muscle stimulation, a second frequency (or range of frequencies) for electrical nerve stimulation, and a third frequency (or range of frequencies) for electrical nerve inhibition.

In various embodiments, any of the neuromodulation devices 100 disclosed herein may be integrated into a number of different form factors and/or incorporated to garments or other fasteners to secure the device 100 with respect to the user's body. For example, a lumbar support belt can be configured to carry the modulation components 107 (e.g., electrodes 109 and/or focused ultrasound transducers 111) such that when the belt is wrapped around the user's waist, the modulation components 107 are directed towards the treatment site of interest. As another example, a posture support belt can be equipped with modulation components 017 such that, when worn by the user, the modulation components 107 are directed towards the treatment site of interest, for example for treating type 2 diabetes. As an additional benefit, such lumbar support belts or posture support belts may be configured to take anatomic pressure off the targeted nerves, for example by pulling the user's shoulder blades back, by exerting pressure on the user's lower back, or by other mechanisms.

In some embodiments, one or more devices 100 can be fastened to a posture support brace in conjunction with a standalone handheld focused ultrasound device to provide therapy to the thoracic region (e.g., to treat type 2 diabetes). In some embodiments, the ultrasound device can be held in place by the user or may be temporarily or permanently coupled to the fastener (e.g., belt, garment, etc.). In a similar way, the one or more devices 100 may be fastened to a lumbar support belt and used to apply therapy to the lumbar region for type-2 diabetes or back pain. Similarly, one or more devices 100 may be fastened to an undergarment and used for treatment of urinary incontinence.

As another example, a knee brace (or elbow brace, ankle brace, etc.) can be equipped to carry one or more modulation components 107 (e.g., electrodes 109 and/or FUS transducer 111). In some instances, it can be disadvantageous to stimulate muscles adjacent the knee as it would inhibit a user's ability to walk. As such, in some embodiments the modulation components 107 coupled to a knee brace can be configured to deliver currents configured to inhibit nerve firing, which is less likely to disturb muscles associated with coordination and walking.

### III. Example Methods of Use

### Mechanism of Action

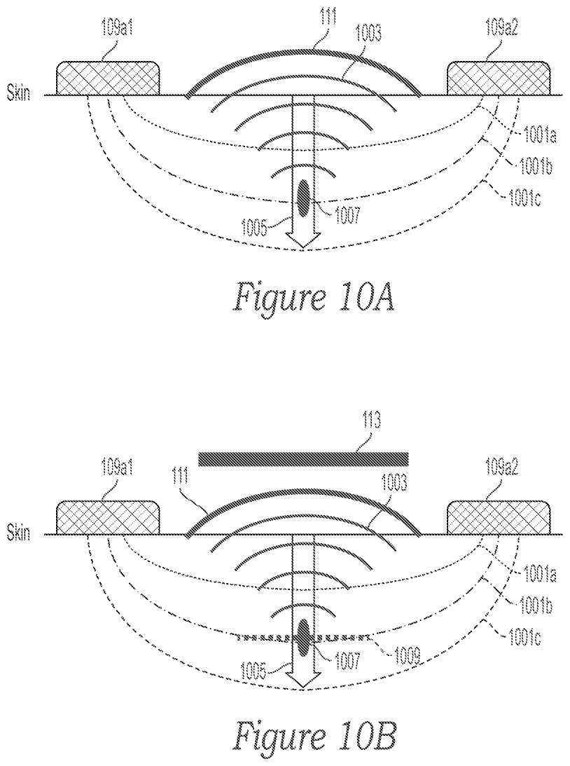

FIG. 10A illustrates a schematic side view of a neuromodulation device delivering electrical and ultrasonic energy to a treatment site. In the illustrated embodiment, the device is positioned over a patient's skin, with first and second electrode bipole pairs 109a1 and 109a2 spaced apart from one another across the skin. Current paths between the bipole electrode pair 109a1-a2 is shown as lines 1001a, 1001b, and 1001c, which reflects current passing at different depths through the tissue beneath the patient's skin. Depending on the particular biological tissue, the drive current applied to the electrodes 109, and other factors, more or less current may travel along any one of the current paths 1001a-1001c. In general, more current will pass along the uppermost path (e.g., current path 1001a), with the amount of current decreasing as the path moves deeper underneath the skin.

The FUS transducer 111 is positioned over the patient's skin at a position between the electrodes 109a1 and 109a2 of the bipole electrode pair. The FUS transducer 111 can be configured to direct an ultrasound beam 1003 along an axis 1005 towards the treatment site 1007. In the illustrated example, the treatment site 1007 is an ellipsoid region beneath the patient's skin and can correspond substantially to the focal zone of the ultrasound beam 1003. In operation, the ultrasound beam 1003 will tend to increase the temperature of the tissue, particularly at or near the focal zone. Increasing the temperature reduces the electrical impedance of the tissue in this region, thereby “steering” current from the electrodes (e.g., current passing along one or more of the current paths 1001a-1001c) into and through the treatment site 1007. When the treatment site 1007 overlaps with a target nerve, the FUS beam 1003 can increase the temperature of at the nerve and/or adjacent tissue, thereby increasing the amount of electrical current passing through the nerve, and increasing the electrical modulation of the target nerve at the treatment site.

In some embodiments, the FUS beam 1003 can be configured and applied so as to raise the temperature of the treatment site 1007 by between about 1-15° F. or about 1-9° C. In some embodiments, the electrodes 109a1 and 109a2 are driven with a frequency of between about 28-100 kHz. Within this range, impedance in biological tissue drops significantly as temperature increases. At higher frequencies (e.g., about 100 kHz), increased temperature may have less effect in reducing impedance, thereby reducing the “steering” effect of the FUS beam 1003.

As noted previously, the focal zone of the FUS beam 1003 can be varied based on the frequency, the geometry of the FUS transducer 111, and other parameters. In various embodiments, the focal zone of the FUS beam 1003 can be selected to correspond to the size of anatomical targets such as a target nerve. By providing a focal zone beneath the surface, electrical current can be steered away from the shallower tissue and towards the treatment site 1007, which may be several millimeters beneath the surface. For example, in the case of targeting nerves of the spine, the treatment site 1007 may be approximately 25-90 mm beneath the patient's skin.

The illustrated bipole electrode pair 109a1-109a2 represents one pair aligned along a first axis of the device. However, as noted previously, in various embodiments there may be two, three, four, or more additional electrode bipole pairs arranged along different axes, each with their own current paths passing through the tissue. In some embodiments, the current paths between these different electrodes also intersects at the treatment site 1007, thereby increasing the total amount of current at the treatment site 1007. In addition to increasing the total amount of current, the different axes provide different radial contributions to the electrical current at the treatment site 1007. This can be useful for modulating nerves positioned in different orientations with respect to the treatment site 1007.

FIG. 10B illustrates a schematic side view of another neuromodulation device delivering electrical and ultrasonic energy to a treatment site. This device can operate similar to that described with respect to FIG. 10A, except that an additional EM coil 113 is provided (shown schematically). As described previously, the EM coil 113 can be driven with alternating current such that a circular electrical current 1009 is induced at a depth below the patient's skin. The induced current 1009 can be arranged in a circular fashion that lies in the same plane as the EM coil 113. In some embodiments, this can be substantially parallel to the plane of the patient's skin. In the illustrated embodiment, the induced electrical current 1009 intersects the treatment site 1007, thereby further contributing to the electrical current passing through the treatment site 1007 and increasing the neuromodulation effects of the device.

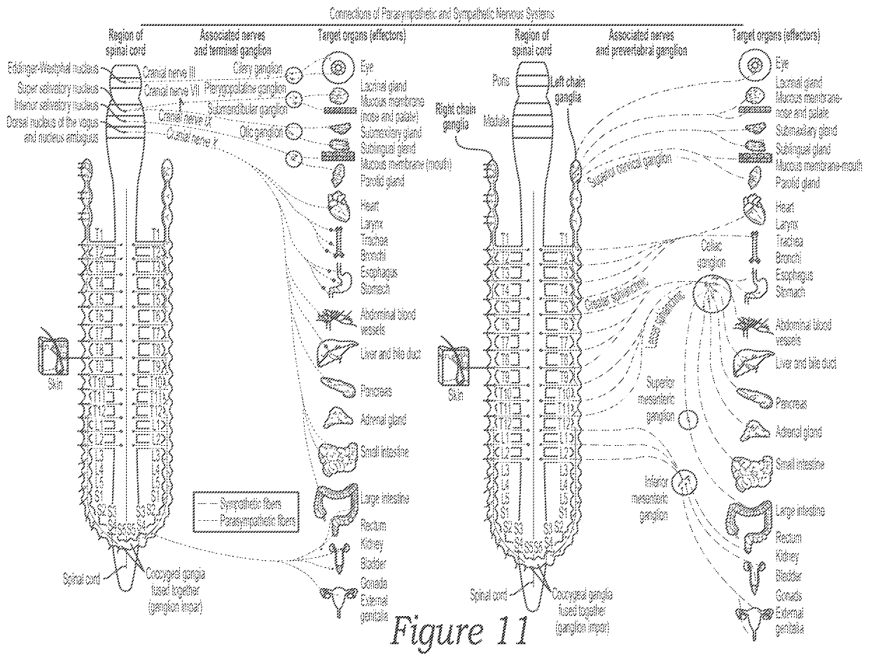

FIG. 11 illustrates anatomical targets for noninvasive modulation of the parasympathetic and sympathetic nervous systems. As illustrated, various sympathetic nerve fibers and parasympathetic nerve fibers are positioned at different regions of the spine. To target modulation of any particular nerve (e.g., for stimulation (up-regulation) or inhibition (down-regulation)), a neuromodulation device 100 can be positioned over the patient's skin at or near the corresponding region of the spine. For example, to modulate activity of the liver, the splanchnic nerves may be targeted by placing a neuromodulation device 100 over the patient's skin adjacent to the T5-T12 region. To treat back pain of a patient, the spinal cord may be targeted at the level of the pain, e.g., at L3. In some embodiments, bilateral treatment can be carried out via use of two devices 100, one on either side of the spine. In other embodiments, a single device 100 may be used to delivery energy to a single treatment site at a time.

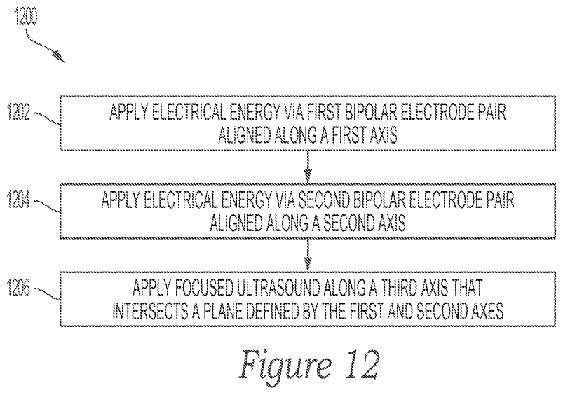

FIG. 12 is a flow diagram of a method 1200 for noninvasively modulating one or more nerves at a treatment site of a patient. Various embodiments of method 1200 include one or more operations, functions, and actions illustrated by blocks 1202 through 1206. Although the blocks are illustrated in sequential order, these blocks may also be performed in parallel, and/or in a different order than the order disclosed and described herein. Also, the various blocks may be combined into fewer blocks, divided into additional blocks, and/or removed based upon a desired implementation.

The method 1200 begins in block 1202 with applying electrical energy via a first bipolar electrode pair aligned along a first axis. For example, as shown in FIG. 10A, first bipole electrode pairs 109a1 and 109a2 can be configured to apply electrical energy. In block 1204, electrical energy is applied via a second bipole electrode pair aligned along a second axis different from the first axis. For example, the first axis and the second axis may intersect one another and lie substantially in the same plane. In one example, the two axes can be aligned in an “X” configuration, such that all four individual electrodes of the two pairs can lie in the same plane and be placed in contact with skin of a patient. In other embodiments, the electrodes may not be aligned along a single plane, for example in order to accommodate a non-planar portion of skin or other treatment site.

The method 1200 continues in block 1206 with applying a focused ultrasound (FUS) beam along a third axis that intersects a plane defined by the first and second axes. For example, the FUS beam can be applied along an axis that is substantially orthogonal to the first and second axes. In one example, the first and second axes lie in a plane such that they can be arranged over the patient's skin, and the FUS transducer is arranged to deliver a FUS beam along an axis substantially orthogonal to the patient's skin and directed toward a treatment site.

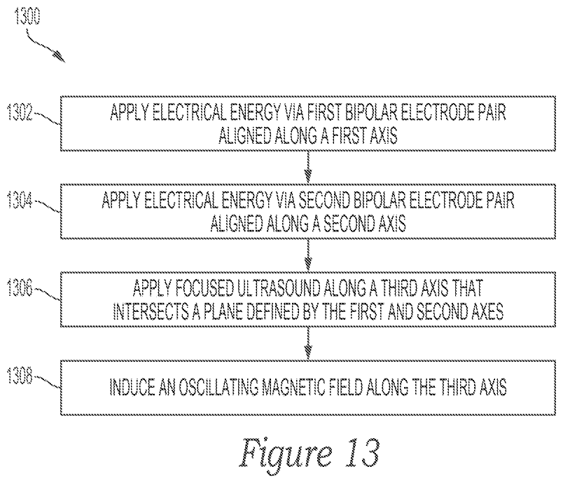

FIG. 13 is a flow diagram of another method 1300 for noninvasively modulating one or more nerves at a treatment site of a patient. Various embodiments of method 1300 include one or more operations, functions, and actions illustrated by blocks 1302 through 1304. Although the blocks are illustrated in sequential order, these blocks may also be performed in parallel, and/or in a different order than the order disclosed and described herein. Also, the various blocks may be combined into fewer blocks, divided into additional blocks, and/or removed based upon a desired implementation.

Blocks 1302 through 1306 of the method 1300 can correspond generally to blocks 1102 through 1106 described above with respect to FIG. 12. Referring to FIG. 13, the method 1300 continues in block 1308 with inducing an oscillating magnetic field along the third axis. For example, an EM coil can be driven with AC such that it induces an oscillating magnetic field that is aligned along an axis substantially orthogonal to the plane of the EM coil. This magnetic field can, in turn, induce a secondary electrical current substantially parallel to the plane of the EM coil. As described above with respect to FIG. 10B, this secondary electric current can be used to deliver additional electrical current to the treatment site.

### Measuring Current Localization

In another aspect of the present technology, a device may be used to measure the electrical current at a treatment site beneath a patient's skin (e.g., as supplied from electrode bipoles at a patient's skin). For example, in the case of a patient undergoing a spinal cord stimulator (SCS) trial, the end of the SCS lead may extend out of the patient's body. The measurement device can be positioned outside of the body and will measure the current on the SCS lead. The SCS lead may be in the epidural space directly midline or either side of midline or place in the area of the dorsal root ganglion. Noninvasive temperature monitoring may be carried out using MM or other suitable technique. Although this measurement technique may usefully provide a measure of electrical current at a treatment site attributable to electrode bipole pairs at a patient's skin, because of the different thermal conductivity of metal compared to surrounding biological tissue, this technique may fail to measure the increased current flow at a treatment site that would be attributable to decreased electrical impedance in tissue as a result of focused ultrasound energy at the treatment site.

### Methods of Guiding System Positioning

In another aspect of the present technology, methods of guiding positioning of a neuromodulation device 100 are provided. For example, a skin fiducial market such as permanent or temporary tattoos (e.g., pigmented to resemble natural skin color and/or freckles) may be put into place to aid patents in identifying the correct position of the neuromodulation device 100 over the skin. For example, for type 2 diabetes patients, the C7 vertebrae may be felt at the back of the neck, and may be used as an anatomical landmark to help patients identify the position of the T5-T9 vertebrae. Similarly, for pain patients, the iliac crest of the hip bone is an anatomical landmark that may help patients identify the position of the L2-L4 vertebrae. Once these positions are identified, a skin fiducial marker can be positioned over desired treatment site, allowing for more reliable and consistent placement of the device 100 for treatment.

### Pain Management

Embodiments of the neuromodulation device 100 can be useful in the treatment of pain, whether acute, chronic, superficial or neuropathic. In particular, chronic neuropathic pain can be treated by inhibition of overactive neurons in and around the spinal cord. In some embodiments, two bilaterally positioned neuromodulation devices 100 can target the dorsal root ganglions on either side of the spinal cord at a particular spine region. In other embodiments, a single neuromodulation device 100 can be positioned over the midline and target the spinal cord directly.

The target site can vary depending on the particular pain being treated. For example, in instances of chronic pain, upper limb and neck pain can be treated by cervical placement of one or more devices, while abdominal pain can be treated by higher thoracic placement. Back pain may be treated with lower thoracic placement of one or more devices, while leg pain can be treated via lumbar placement. For dorsal root ganglion therapy, the device(s) 100 may be placed at the level of pain, for example between L2-L4.

The devices and methods disclosed herein may also be used to treat unilateral or bilateral pain associated with one or more of the following: failed back syndrome (FBS) or low back syndrome or failed back, radicular pain syndrome or radiculopathies resulting in pain secondary to FBS or herniated disk, postlaminectomy pain, multiple back operations, unsuccessful disk surgery, degenerative disk disease (DDD), peripheral causalgia, epidural fibrosis, arachnoiditis, reflex sympathetic dystrophy, or multiple sclerosis related pain

As noted previously, one advantage of the devices and methods disclosed herein is that electrical current is delivered to a treatment site along multiple radial directions. Since the target nerves have varying neuroanatomical orientation and complex shapes, delivering current along multiple axes may increase therapeutic benefit by increasing the likelihood of current aligning with the target anatomy. In contrast, conventional spinal cord stimulation typically delivers current along a single axis only (e.g., with one bipole). As a result, with no current flowing perpendicular to the single bipole pair, therapeutic benefit may be limited.

In various embodiments, the timing, dosing, and treatment regimen may vary from patient to patient. In some embodiments, treatment locations may cycle between T9 and L2-L4, with the T9 position focusing on dorsal root ganglion inhibition and L2-L4 focusing on therapeutic ultrasound to the anatomical insult. In some embodiments, the electrodes may be used along without focused ultrasound throughout the day or while a patient is mobile, with the focused ultrasound being used in conjunction with the electrodes only when an external power source is available. In one example, a patient may use one 30-minute treatment of electrical and focused ultrasound energy per day, or two 15-minute sessions per day, one targeting T9 and the other targeting L2-L4.

### Type II Diabetes

Sympathetic hyperactivity activity is strongly associated with Type II diabetes. Autonomic control of hepatic glycogen production likely occurs through the sympathetic vasoconstriction or parasympathetic vasodilation of the hepatic sinusoids causing either a glycogenolytic or glycogenic cascade.

Approximately two-thirds of patients with poorly controlled type 2 diabetes have abnormal gastric emptying that is mostly asymptomatic. Approximately 30% of patients with type 2 diabetes have diabetic gastroparesis, for which roughly 50% of these patients have corresponding abdominal pain. A rodent study suggests that insulin resistance and glucose intolerance can be improved through chronic intestinal electrical modulation through a proposed mechanism involving increasing incretin release, including GLP-1 and GIP, and reducing gastric ghrelin release. High amplitude modulation coupled with ON/OFF duty cycling in a way to normalize GI slow waves are believed to be the most effective SCS parameters to promote GI motility.

GI tract dysmotility, hypoperfusion, and loss of luminal barrier integrity are relevant to the development of systemic inflammation. Cytokine panels for patients with type 2 diabetes differ from normal and are involved in type 2 diabetes development as well as disease progression. Systemic cytokines have been associated with the development of chronic back pain. Additionally, spinal cord stimulation (SCS) and sacral nerve stimulation with duty cycle have each been demonstrated to optimize inflammatory cytokines. Modulation parameters that normalize GI slow waves may optimize cytokine panels in patients with type 2 diabetes and may more effectively treat pain disorders as well.

In various embodiments, a neuromodulation device 100 as disclosed herein may be used to apply electrical and/or ultrasound energy to a treatment site for a patient with type 2 diabetes. In particular, the device 100 may be used to inhibit the hepatic splanchnic nerves and other associated sympathetic nerves, thereby allowing the liver to update more glucose. Increased glucose uptake by the liver reduces the glucose in a patient's bloodstream (e.g., as measured by the curve during an oral glucose tolerance test (OGTT)). Such splanchnic inhibition may also lower a type 2 diabetes patient's fasting glucose. Accordingly, treatment modalities for type 2 diabetes may include modulation around mealtimes to reduce glucose spikes from meals, which over time may decrease a patient's average serum glucose over time (e.g., as measured via HbA1c % values over time).

In some embodiments, the treatment site of the device 100 (e.g., corresponding to the focal zone of the FUS transducer 111) can correspond to the hepatic splanchnic nerve to provide the desired inhibition, for example via relatively high frequency (e.g., kHz frequency ranges). For example, the device 100 can be placed over a patient's skin adjacent to the splanchnic nerve regions from around T5-T12. In some embodiments, it can be advantageous to avoid inhibition of the vagal nerve and its branches that are anterior to the spinal cord, which innervate the liver, stomach, spleen, pancreas, and small and large intestines. This may be particularly important because modulation of the vagal nerve has been used to treat type 2 diabetes (e.g., lowering OGTT), and therefore inhibition of the vagal nerve may have deleterious effects for patients with type 2 diabetes. Because the combination of focused ultrasound and electrical energy provides a small treatment site and focuses electrical energy into the desired region, inhibition of the hepatic splanchnic nerve can be achieved without also inhibiting the vagal nerve and its branches.

In some embodiments, the neuromodulation device 100 can be coupled to a blood glucose monitor (e.g., the device 100 may incorporate a blood glucose monitor or it may communicate wirelessly or otherwise with an external blood glucose monitor). In some embodiments, the device 100 can vary the delivery parameters for electrical and/or ultrasound energy based on measurements taken by the blood glucose monitor. For example, during hyperglycemic events, a blood glucose monitor may detect high glucose levels. In response, the neuromodulation device 100 may begin delivery of electrical and ultrasound energy to a treatment site (e.g., the hepatic splanchnic nerve). In one example, kHz range electrical current is applied to the hepatic splanchnic nerve, thereby inhibiting activity of the nerve and increasing hepatic glucose uptake in the patient. Once glucose levels have returned to a normal or acceptable range as determined by the blood glucose monitor, the neuromodulation device 100 may cease delivery of electrical and ultrasound energy to the treatment site.

In some embodiments, the device 100 may be used to increase blood glucose levels in response to hypoglycemia. For example, increasing the temperature of splanchnic nerves with ultrasound without high frequency electrical inhibition might activate nerves and increase blood glucose. Accordingly, applying only ultrasound energy to the splanchnic nerve without any concurrent electrical energy can increase activity of the nerve (rather than inhibit activity), thereby reducing hepatic uptake of glycogen, and increasing glycogen levels in the blood. This permits the device 100 to provide closed loop control over hepatic glycogen by increasing glucose when blood sugar is low (e.g., by using ultrasound and electrical energy to inhibit the splanchnic nerve) and decreasing glucose when it is high (e.g., by using ultrasound only to increase splanchnic nerve activity).

In some embodiments, the device 100 may vary one or more parameters of the delivered energy depending on the glucose measurements, rather than simply cycling on/off. For example, the device 100 may supply ultrasound energy only (e.g., via the FUS transducer 111) in one state, while supplying both ultrasound and electrical energy (e.g., via the FUS transducer 111 and the electrodes 107 simultaneously) in another state. In another example, the device 100 may vary the applied electrical frequency depending on the measured glucose level.

In some embodiments, to effectively treat a patient with type 2 diabetes, it can be useful to apply broad sympathetic inhibition across a larger treatment region as opposed to narrowly targeting the splanchnic nerve. The T5-10 spinal region involves the liver, adrenals, pancreas, and GI tract, which are all organs involved in type 2 diabetes. Short-term energy maintenance occurs through glycogen production and release from the liver. The liver is directly modulated the autonomic tone delivered to the liver as well as by pancreatic, adrenal, and GI hormones including insulin, glucagon, catecholamines, GLP-1, and GIP. Accordingly, broad sympathetic inhibition across this region may promote glycogen production in the liver through promoting insulin and incretin (GLP-1 & GIP) release, and inhibiting glucagon and catecholamine release. Further, inhibiting the sympathetic tone directly innervating the liver may vasodilate hepatic vessels and cells triggering a glycogenic biochemical cascade. A device 100 as shown in FIG. 7 can be particularly useful in applying broad sympathetic inhibition across the T5-T10 region to treat type 2 diabetes. This broad inhibition may reduce initial glucose spikes as more glucose is channeled into the liver, while also promoting more rapid return of serum glucose levels to baseline. This inhibition may also reduce fasting blood glucose levels and improve inflammatory cytokine profiles for patients with type 2 diabetes. Further, this broad inhibition may treat diabetic gastroparesis symptoms by improving GI motility.

In treating patient's with type 2 diabetes, it can be useful to control the timing, duty cycle, and other such parameters of the treatment device 100 to most effectively control blood glucose levels. For example, it can be useful to time delivery of therapeutic energy to the treatment site around mealtimes. For example, post-prandial glycemic spikes are the primary source of hyperglycemia. Accordingly, prior to eating and at least some length of time into a meal (e.g., 15 minutes prior to eating and 15 minutes into a meal), it can be useful to apply both electrical and ultrasound energy to the treatment site (e.g., the splanchnic nerve). As described above, this promotes hepatic uptake of glucose and conversion to glycogen. During non-meal times, the device may be used to synch stomach slow wave rhythms, for example by using electrical energy only (without concurrent ultrasound), for example 20 seconds on, followed by 20 seconds off.

For example, it can be beneficial to normalize slow waves of the gastrointestinal region of interest by syncing duty cycle of electrical energy delivered via the device with healthy physiological slow wave GI rhythms through proposed sympathetic inhibition. In some embodiments, to normalize slow waves of a GI region of interest, the device 100 applies only electrical energy without concurrent ultrasound. In one example, when targeting the stomach, the device may be powered on for 20 seconds, then off for 20 seconds to normalize to 3 waves per minute slow wave frequency. To target the duodenum, the device 100 may be powered on for 5 seconds, then off for 5 seconds to normalize to 12 waves per minute slow wave frequency. To target the jejunum, the device 100 may be powered on for 5.5 seconds, then off for 5.5 seconds to normalize to 11 waves per minute slow wave frequency. The ileum can be targeted by powering the device on for 7.5 seconds, then off for 7.5 seconds to normalize to 8 waves per minute slow wave frequency. Similarly, the large intestine can be targeted by powering the device 100 on for 10 seconds, then off for 10 seconds to normalize to 6 waves per minute.

In some embodiments, for patients with type 2 diabetes, the device 100 may be turned off in the pre-dawn hours (e.g., between 1:00 am-4:00 am) to prevent hypoglycemic Somogyi effects and the dawn phenomenon. These effects may stem from patients taking insulin before bed causing a hypoglycemic event, which then triggers a resulting sympathetic response resulting in a hyperglycemic episode early the next morning.

### Urinary Incontinence

The devices and methods disclosed herein may be applied to treatment of urinary incontinence. For example, the neuromodulation device 100 can be applied over a patient's skin to target sacral nerves such as the pelvic splanchnic nerve (S2-S4) and/or lumbar splanchnic (L1/L2) nerves to inhibit activity in these target nerves. For example, the device 100 can be positioned such that the treatment site is at or near one or more of the sacral foramen such that one or more of the sacral nerves at stimulated via the combination of ultrasound and electrical energy supplied by the device. This modulation can inhibit activity in the sacral nerves, thereby reducing or eliminating urinary incontinence. Similar approaches may be utilized in treatment of fecal incontinence. In some embodiments, a neuromodulation device 100 as disclosed herein can be used to treat overactive bladder syndrome, for example via tibial nerve modulation.

### Migraine

The devices and methods disclosed herein may be applied to treatment of migraines. In some embodiments, a device 100 as shown in FIG. 8 can be particularly suited for treatment of migraines. For example, the neuromodulation device 100 can be applied over the back of a patient's neck to target nerves stemming from the cervical region including the occipital nerves. Currently available treatments for migraine relief include surgical decompression of the greater occipital nerve in the cervical area as well as are Botox injections in the cervical region, which inhibit overactive nerves in this area. The device 100 may provide involve one or two electric bipoles (e.g., electrodes 109a1-a2 and 109b1-b2 shown in FIG. 8) coupled with superficial electric heating, which could suffice in place of focused ultrasound as pertinent nerves are more superficial around the back of the neck. The high frequency (e.g., kHz range) electrical currents may inhibit overactive nerves, analogous to surgical decompression or Botox injections in the same region. The device 100 can be configured to be placed around a user's neck or over a user's shoulders such that the electrodes contact the back of the patient's neck, as seen in FIG. 8.

### Cancer

The devices and methods disclosed herein may be applied to treatment of cancer. For example, the neuromodulation device 100 can be applied over the head to treat glioblastoma. In some embodiments, high-frequency electrical currents (e.g., in the kHz range) in multiple planes can penetrate deeply to brain targets and the focused ultrasound can focus the current to brain tumor targets as FUS can penetrate through bony structures including the skull. This would be an improvement to current high-frequency electrical current therapies for cancer, as focused ultrasound may improve focus of the electrical current to the desired therapeutic targets. Other cancer indications include mesothelioma, non-small cell lung cancer, pancreatic cancer, ovarian cancer, and liver cancer.

### Other Clinical Indications

Various embodiments of the devices disclosed herein may likewise be used to treat other clinical indications. For example, any clinical indication associated with sympathetic hyperactivity may be treated by using a neuromodulation device as disclosed herein to delivery electrical and/or ultrasound energy towards a target nerve or treatment site. Embodiments of the devices disclosed herein can be used to treat one or more of: gastroparesis, inflammatory bowel disease (IBD), rheumatoid arthritis (e.g., targeting the vagus nerve), spinal cord injuries, osteoarthritis, movement disorders (e.g., Parkinson's disease, essential tremor, and dystonia) via modulation of deep brain structures such as subthalamic nucleus (STN), ventral intermediate nucleus (Vim), or thalamus. Additional indications that may be effectively treated by applying ultrasound and electrical energy as disclosed herein dementia (targeting the hippocampus), autism (targeting the arcuate fasciculus), inflammatory disorders (e.g., targeting fibers innervating the spleen and GI tract), obesity (e.g., VNS block), multiple sclerosis, Crohn's disease, ulcerative colitis, lupus. Skin conditions may also be treated using the devices and methods disclosed here, for example improving aesthetics or treating various skin pathologies including cancer.

### III. Conclusion

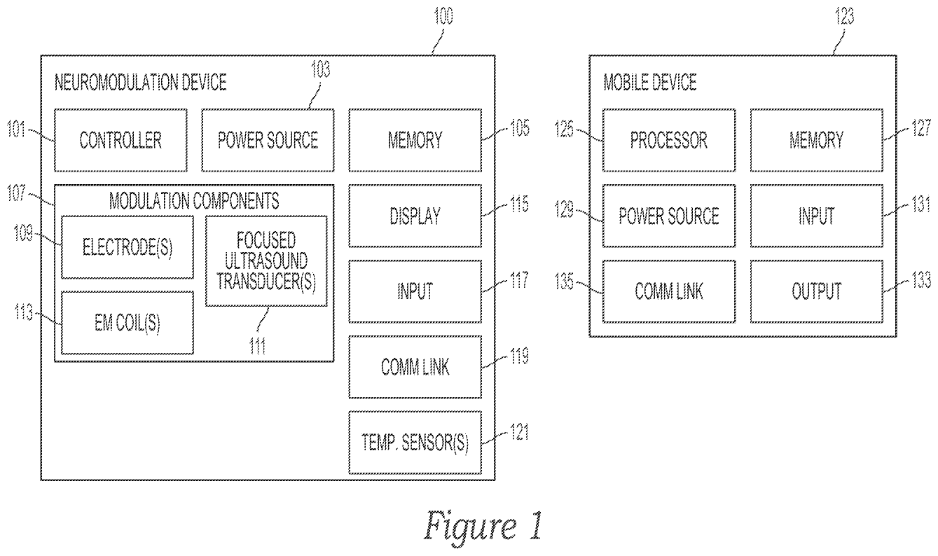

Although many of the embodiments are described above with respect to systems, devices, and methods for noninvasive neuromodulation, the technology is applicable to other applications and/or other approaches, such as noninvasive application of electrical and/or ultrasonic energy for other non-neuromodulation applications. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-13.

The above detailed descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Where a range of values is provided, it is understood that each intervening value, to the tenth of the unit of the lower limit unless the context clearly dictates otherwise, to between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-77. (canceled)

78. A noninvasive electrical treatment device comprising:
a first bipole electrode pair aligned along a first axis;
a second bipole electrode pair aligned along a second axis, the first axis and the second axis defining a plane;
a focused ultrasound (FUS) transducer configured to direct a focused ultrasound beam along a third axis that intersects the plane;
a controller electrically coupled to the first and second bipole electrode pairs and to the focused ultrasound transducer, the controller configured to:
apply electrical energy having a frequency of between about 1 Hz to about 100 MHz to the first and second bipole electrode pairs; and
cause the FUS transducer to emit a focused ultrasound beam having a frequency of between about 20 kHz to about 10 MHz.

79. The device of claim 78, wherein the device is configured to be applied over a patient's head to apply the electrical energy to a treatment site in the patient's brain.

80. The device of claim 78, wherein the third axis intersects the plane at an angle greater than 45 degrees.

81. The device of claim 78, further comprising a third bipole electrode pair aligned along a fourth axis, wherein the controller is configured to apply electrical energy having a frequency of between about 1 Hz to about 100 MHz to the third bipole electrode pair.

82. The device of claim 78, wherein the first bipole electrode pair comprises first and second electrodes separated by a distance of between about 3-35 cm.

83. The device of claim 78, wherein the FUS transducer is a spherical FUS transducer.

84. The device of claim 78, wherein the spherical FUS transducer has a radius of curvature of between about 10-150 mm.

85. The device of claim 78, further comprising a temperature sensor electrically coupled to the controller, and wherein the controller, in response to an indication that the temperature sensor has detected a temperature above a predetermined threshold, is configured to:
cease applying electrical energy to the first and second bipole pairs; and/or
cease causing the FUS transducer to direct the focused ultrasound beam.

86. A device comprising:
an array of electrodes disposed around a central region, the electrodes configured to apply electrical current transcutaneously to a treatment site;
a focused ultrasound (FUS) transducer disposed in the central region, the FUS transducer configured to apply focused ultrasonic energy to the treatment site; and
a controller electrically coupled to the array of electrodes and to the FUS transducer, the controller configured to apply electrical energy to the array of electrodes and to cause the FUS transducer to emit a focused ultrasound beam.

87. The device of claim 86, wherein the device is configured to be applied over a patient's head to apply the electrical current to a treatment site in the patient's brain.

88. The device of claim 86, wherein the array of electrodes comprises at least four electrodes arranged circumferentially around the central region.

89. The device of claim 86, wherein the array of electrodes comprises a plurality of bipole electrode pairs, with individual electrodes of each bipole electrode pair separated from one another by the central region.

90. The device of claim 86, wherein the FUS transducer is a spherical FUS transducer.

91. A method comprising:
applying electrical energy to a plurality of bipole electrode pairs disposed over a patient's skin proximate to the treatment site such that current flowing between each of the bipole electrode pairs intersects at or adjacent to the treatment site; and
applying a focused ultrasound beam through the patient's skin and towards the treatment site.

92. The method of claim 91, wherein applying the focused ultrasound beam through the patient's skin and towards the treatment site comprises heating the treatment site.

93. The method of claim 91, wherein the electrode pairs are disposed over the patient's head.

94. The method of claim 91, wherein heating the treatment site lowers electrical impedance at the treatment site, thereby increasing electrical current density at the treatment site.

95. The method of claim 91, wherein the treatment site comprises a brain region of the patient.

96. The method of claim 95 wherein the treatment site comprises a glioblastoma in the brain region.

97. The method of claim 91, wherein the electrical energy comprises alternating current having a frequency of between about 1 Hz to about 100 MHz.
